(19) United States
(12) Patent Application Publication
(10) Pub. No.: US 2025/0101089 A1
(43) Pub. Date: Mar. 27, 2025

(54) TAU THERAPY

(71) Applicants: CAMBRIDGE ENTERPRISE LIMITED, Cambridge (GB); UNITED KINGDOM RESEARCH AND INNOVATION, Wiltshire (GB); AHREN LP, ACTING BY ITS GENERAL PARTNER AHREN INNOVATION CAPITAL GUERNSEY (GP) LIMITED, Cambridge (GB)

(72) Inventors: William Alexander MCEWAN, Cambridge (GB); Aamir Shehab MUKADAM, Cambridge (GB); Lauren Virginia Clare MILLER, Cambridge (GB); Benjamin James TUCK, Cambridge (GB); Sophie Elizabeth KEELING, Cambridge (GB); Annabel Emily SMITH, Cambridge (GB); Gregory Paul WINTER, Cambridge (GB); Leo C. JAMES, Cambridge (GB)

(21) Appl. No.: 18/832,480

(22) PCT Filed: Jan. 24, 2023
(86) PCT No.: PCT/EP2023/051711

§ 371 Date: Jul. 23, 2024

(30) Foreign Application Priority Data
Jan. 24, 2022 (GB) 2200872.6
Aug. 17, 2022 (GB) 2212019.0

(51) Int. Cl. C07K 16/18; A61K 39/00; A61P 25/28

(52) U.S. Cl. CPC C07K 16/18; A61P 25/28; A61K 2039/505; A61K 2039/54; C07K 2317/53; C07K 2317/71; C07K 2317/72; C07K 2317/76; C07K 2317/82; C07K 2317/92

(57) ABSTRACT

The invention provides a ligand comprising a first binding moiety which binds to tau assemblies, and a second binding moiety which is bound by TRIM21 for use in the treatment of neurodegenerative disease in the cytoplasm of a neuronal cell, wherein the ligand is administered extracellularly.

The present application relates to the use of anti-tau antibodies to target tau protein aggregates which are involved in the pathogenesis of dementia and neurodegenerative disease, including Alzheimer's disease. In particular, we demonstrate that tau-specific antibodies do not block the entry of tau to neurons but act post-entry via the action of the cytoplasmic tripartite motif-containing protein 21 (TRIM21; Trim21; T21). The invention therefore provides tau therapy based on administration of ligands which bind to tau assemblies and to TRIM21.

Antibody-mediated immunity forms a crucial part of the anti-pathogen immune response. Antibody-mediated neutralization is generally considered to occur extracellularly due to exclusion of antibody from the cell interior by membrane compartmentalization. Extracellular neutralization has also been reported to be potentiated by engagement of Fc receptors (Bournazos, 2014; DiLillo, 2014).

Extracellular viral neutralization is thought to require a given level of antibody occupancy of specific epitopes to prevent entry into cells (Klasse 2002). However, viruses are known to display immuno-dominant epitopes that bias the polyclonal antibody response toward epitopes that do not block viral entry (Leung 2004; Sumida 2005; Schrader 2007). Some viruses and bacteria have the capacity to penetrate the cell membrane and enter the cytosolic compartment even when they are opsonized with antibody.

Recently, a novel mechanism termed antibody-dependent intracellular neutralization (ADIN) was described where it was shown that antibodies can mediate neutralization intracellularly by recruiting a cytosolic antibody binding receptor named TRIM21. The engagement of antibody-virus complexes by TRIM21 promotes the degradation of both antibodies and virus by the proteasome.

Antibody-bound pathogens entering the cytoplasm are rapidly sensed by cytosolic TRIM21, which induces a synchronized effector and signalling response. Antibody-opsonized non-enveloped viruses are rapidly targeted for degradation via the proteasome and induce an innate immune response. Bacteria in complex with antibody trigger innate immune signalling (McEwan 2013) and possibly killing via autophagy (Rakebrandt 2014).

In both cases, TRIM21 functions as a link between the intrinsic cellular self-defence system and adaptive immunity by taking advantage of the diversity of the antibody repertoire to detect invaders (Randow 2013). By doing so, TRIM21 distinguishes itself from other members of the TRIM protein family with anti-viral functions as these generally recognize the invading pathogen directly (Bottermann 2018). TRIM21 recognition is also distinct from that of other innate sensors, such as pattern recognition receptors, which detects pathogen associated molecular patterns (PAMPs) (Odendall 2017). Instead, TRIM21 treats the displacement of antibody from the extracellular to the intracellular environment as a danger associated molecular pattern (DAMP) (McEwan 2013). Viral restriction by TRIM21 may also synergize with protective anti-viral mechanisms mediated by the complement system (Tam 2014, Bottermann 2019).

Many neurodegenerative disorders are characterized by aggregation of an intracellular protein, such as the intracellular microtubule-associated tau protein in Alzheimer's disease (McEwan 2017). Intracellular tau can be induced to aggregate by extracellular tau aggregates, sometimes called seeds, that can spread between cells in the manner reminiscent of a virus or prion (Frost 2009; Guo 2011). Administration of tau specific antibody is known to reduce tau pathology in mice (Yanamandra 2013; Asuni 2007; Chai 2011; Sankaranarayanan 2015), but the underlying mechanism is not fully elucidated.

Attempts to treat tau pathology with anti-tau antibodies have met with limited success. It remains unclear which mechanisms of action operate in vivo. As described in Beltran and Sigurdsson, (2020) Neuropharmacology 175; 108104, incorporated herein by reference, the majority of clinical interventions appear to attempt tau clearance using antibodies, either extracellularly by blocking tau entry into neurons or intracellularly by causing their degradation. Different antibodies appear to have differing properties regarding entry into neurons; those that do enter into the cell appear to require FcγRs for entry. Moreover, it is unclear whether intracellular antibodies are cytosolic or endolysosomal in action. Overall, despite emerging evidence of clinical efficacy no clinical approach to tau therapy using anti-tau antibodies has yet been approved by the FDA.

Antibodies that bind to tau may bind to monomeric tau or to tau in its various conformational states, including oligomeric tau and the diverse fibrillar forms of tau that have been isolated from human brains (Shi et al., 2021). Additionally, antibodies may bind to post-translationally modified forms of tau including phosphorylated, acetylated, truncated and otherwise-modified tau variants. For the purposes of this application, we consider antibodies that bind to assembled forms of tau. These may therefore include antibodies that bind to tau monomer and phosphorylated tau sites, both exemplified herein, as well as conformation-specific antibodies and antibodies specific for other modifications present within tau assemblies.

Interestingly, TRIM21 has been shown to inhibit intracellular tau aggregation in an cells based tau seeding assay (McEwan, 2013). Seeded propagation of misfolded tau is proposed to underlie many common neurodegenerative disorders. Akin to viral infection, this model of seeded tau propagation relies on the physical transfer of tau assemblies between cells, and is therefore potentially susceptible to interception by antibody.

However, the tau seeding assay relies on administration of tau seeds together with anti-tau antibodies by transfection into cells and does not show that anti-tau antibodies administered extracellularly, and without using transfections methods, would be able to enter neurons bound to tau aggregates or seeds, engage TRIM21, and successfully destroy such aggregates. It is thus a demonstration of the potential of anti-tau antibodies to target tau aggregates intracellularly but does not establish that such a mechanism can operate successfully in vivo.

Moreover, work to date has not established whether extracellular neutralisation or intracellular degradation is the prevailing mechanism in vivo.

Kondo et al. (215) Nature 523; 431 shows an anti-tau antibody that protects against a cis-isomer of tau. Knockdown of Trim21 reduces its protective effect but how, and where in the cell, is unknown. This takes on significance as Trim21 has been shown to be expressed on the cell surface as well as in the cytosol (Hillen et al., 2020; Miranda et al., 1998). Moreover, the cis-tau target of the antibody is distinct as a therapeutic target from the cytosolic aggregates that accumulate in neurodegenerative disease.

There remains the need, therefore, for a therapeutic approach to the treatment of tau pathology in dementia and other neurodegenerative diseases which is applicable to a subject in vivo.

We demonstrate herein that cells internalise anti-tau ligands together with tau aggregates, and that the anti-tau ligands co-internalised with tau successfully neutralise the tau aggregates through the activity of Trim21. We also demonstrate viral neutralisation following co-internalisation with viruses and anti-viral antibodies. Based on the data presented herein, the present invention provides a ligand that simultaneously binds to tau assemblies and to TRIM21. Where the ligand in question is an antibody, our invention advantageously incorporates a combination of engineered modifications to the antibody that promotes increased TRIM21 activity, preserves or enhances antibody half-life by maintaining or increasing FcRn binding compared to an unmodified antibody, and reduces pro-inflammatory effects by reducing or removing interactions with FcγRs and complement.

In addition to antibodies, other ligands can be used to selectively bind tau assemblies. Several positron emission tomography (PET) ligands have been developed, described in Leuzy et al 2019 and incorporated herein by reference (Leuzy et al., 2019). Such compounds can be conjugated to ligands that bind to ubiquitin E2 or E3 enzymes, generating bispecific binding molecules, PROTACs. The PET ligand AV1415 was coupled to a ligand that binds the E3 ubiquitin ligase cereblon and successfully promoted degradation of tau assemblies (Silva et al., 2019). Other small molecules and structured peptides can be selected or engineered to bind to tau assemblies. This includes cyclic peptides, linear peptides, peptide mimetics and other small molecules including those originating from fragment-based libraries and screening campaigns such as DNA-encoded libraries. Where conjugated to a ligand that binds TRIM21, these ligands can be used in the current invention.

### SUMMARY OF THE INVENTION

According to a first aspect, the present invention provides a ligand comprising a first binding moiety which binds to tau assemblies, and a second binding moiety which is bound by TRIM21 for use in the treatment of neurodegenerative disease by degrading tau intracellularly in the cytosol of a neuronal cell, wherein the anti-tau ligand is administered extracellularly.

Blocking the entry of tau assemblies is one way in which Abs may operate to prevent seeded aggregation of tau. Tau assemblies can be taken up into neurons via an interaction with heparin sulphate proteoglycans and the low density lipoprotein receptor LRP1 (Holmes et al., 2013; Rauch et al., 2020). The site on tau that is responsible for these interactions is predominantly the repeat region, though the tau N-terminus also plays minor role in contacting LRP1.

There is a longstanding view that the ubiquitin-proteasome system (UPS) is unable to process aggregated proteins such as tau filaments and that macro-autophagy is responsible for degradation of aggregated tau species (Rubinsztein, 2006). In recent years this has been challenged by the discovery that, given the correct adaptor for E3 ligase recruitment, tau assemblies can be selectively degraded in the cytosol by the ubiquitin-proteasome pathway. Examples of such adaptors include antibody, which recruits the E3 ligase TRIM21 to tau assemblies (McEwan et al., 2017a), and PROTACs derived from the tau PET imaging tracer AV1415 which selectively binds assembled versions of tau and recruits the E3 ligase cereblon (Silva et al., 2019). There is accordingly a potential for therapeutic targeting of intracellular tau species in the brain. However, the mechanism by which antibodies, which are predominantly extracellular, might be taken up by the cell leading to protection against intracellular tau pathology remained incompletely understood in the prior art. Before the present invention, it had not been demonstrated that antibody therapy against tau pathology depends on the cytosolic Fc receptor TRIM21.

We show herein that the anti-tau ligand is co-internalised by the cell together with tau. Thus, the ligand does not block tau entry to any therapeutically meaningful extent; on the contrary, the anti-tau ligand is dragged in to the cell cytoplasm though tau uptake by the cell. In the cytoplasm, the anti-tau ligand is effective to neutralise tau by binding to Trim21, leading to degradation of the tau protein.

In some embodiments, the ligand for use according to the first aspect of the invention is administered intravenously to a subject.

Binding of the ligand to tau or tau assemblies can be direct or indirect. Where it is indirect, it may bind to a second ligand which is specific for tau or a tau assembly. Advantageously, the binding is direct and the ligand binds specifically to tau or a tau assembly. Advantageously, the binding is to a tau assembly.

The tau binding domain can take any suitable form, as further described below. For example, the ligand can be a polypeptide, a structured polypeptide, or a small molecule. The ligand is taken up into the cell through association with tau assemblies.

The TRIM21 binding domain of the ligand can also take any suitable form, and can be a polypeptide, structured polypeptide or small molecule. In one embodiment, the TRIM21 binding domain can be an antibody Fc region.

In some embodiments, the ligand does not bind FcγR to any significant degree, or is a ligand in which the ability to bind to FcγR and/or complement (C1q) has been reduced or eliminated. FcγR and/or complement binding is associated with the effector function of antibodies in which antibody-tau complexes would be taken up and degraded by cells expressing FcγRs such as microglia. We have shown that such an activity, though present, is of minor importance in the antibody protection against seeded aggregation. Much more important is the ability of antibodies, once inside the cell, to direct the tau assemblies to the UPS via TRIM21. Therefore, the use of ligands which lack FcγR and/or complement binding carries no substantial disadvantage in terms of the treatment of tau pathology, but are associated with benefits in reducing the inflammatory response to antibody administration in a subject. There is uncertainty in the field surrounding whether therapeutic antibodies against tau should seek to engage FcγRs. On the one hand, engagement of FcγRs promotes internalisation and degradation of extracellular antibody-bound tau assemblies and this mechanism is explicitly used by certain therapeutic antibodies (Andersson et al., 2019; Zilkova et al., 2020). On the other hand, engagement of FcγRs responses may be damaging in the CNS by promoting activation innate immune system. During immunotherapy against beta-amyloid, a comparison of human IgG1 with IgG4 suggested that reduced engagement of FcγRs was associated with lower induction of inflammatory markers (Adolfsson et al., 2012). The present invention relies on TRIM21 for its effector function and therefore FcγR interactions may be reduced or dispensed with, removing the potentially deleterious effects of the inflammatory response without removing effector function.

The ligand is advantageously an antibody which comprises a variable domain which binds to tau or tau assemblies, and a Fc region which is bound by TRIM21.

Advantageously, the antibody is adapted for intracellular activity.

If the TRIM21 binding domain comprises an antibody Fc region, in some embodiments, FcγR binding is reduced by introducing, N297A, L234A and L235A (LALA), or P329G, L234A and L235A (PGLALA), or N297A, L234A and L235A (NALALA) mutations into an IgG1 Fc domain. Binding may also be reduced by inserting mutations such as L234F/L235E/P331S (FES) or L234F/L235E/D265A (FEA). Alternatively, the immunoglobulin Fc fragments can be derived from an immunoglobulin class or isotype that have reduced binding affinity to Fc gamma receptors or complement; for example, human IgG2 or IgG4 Fc domains can be used, as well as their derivatives that further ablate binding (eg IgG4-PE S228P/L235E).

In some embodiments, the antibody is aglycosylated (for example by introducing mutations N297A/G/Q) or deglycolsylated after expression. Loss of glycosylation does not prevent TRIM21 activity.

In some embodiments, the ability of the antibody Fc domain to bind to TRIM21 has been increased, relative to the unmodified antibody. Exemplary mutations include mutations at positions 131, 256, 311, 345, 385, 433, 434, 435, 436 and/or 428. Increasing the binding to TRIM21 increases the effectiveness of the therapeutic effect, as we have shown that this is mediated almost exclusively through TRIM21.

In some embodiments, improved TRIM21 activity can be obtained through other approaches, for example due to changes in the hinge region (for example removal of disulphide bridges) or an antibody in which the binding or activity of TRIM21 is increased by using a different antibody isotype (eg IgG3) or by extending the hinge region.

Mutations in the Fc domain of the antibody can increase affinity for TRIM21; an exemplary mutation is T256P, as well as further mutations as set forth in more detail below.

In some embodiments, antibody recycling via FcRn is enhanced. Preferably, one, two, three or four mutations selected from the group consisting of M252Y, S254T, T256E, H433K and N434F are introduced into the IgG1 Fc domain to enhance FcRn binding. Preferably, a combination of mutations consisting of M428L/N434S or T256D/T307Q (DQ) or T256D/T307W (DW) or M252Y/T256D D (YD) or T307Q/Q311V/A383V or T256D/H286D/T307R/Q311V/A378A or L309D/Q311H/N434S (DHS) or M252Y/S254T/T256E (MST-YTE) is introduced into the IgG1 Fc domain. This makes the antibody more abundant owing to having a longer half-life in circulation.

In a second aspect, there is provided an anti-tau antibody in which the ability to bind to FcγR and/or complement (C1q) has been reduced or eliminated, preferably in complex with tau.

The antibody may be modified as described in the preceding aspect of the invention.

In a third aspect, there is provided an anti-tau antibody in which the ability to bind to TRIM21 though the Fc domain has been increased, for example by incorporating one or more of the antibody mutations T256P, H433T, N434R, Y436F and S4401 to human IgG1 or their equivalents in other IgG Fc domains.

In a fourth aspect, there is provided an anti-tau antibody in which antibody recycling via FcRn is enhanced. Preferably, one, two, three or four mutations selected from the group consisting of M252Y, S254T, T256E, H433K, N434F are introduced into the IgG1 Fc domain to enhance FcRn binding. Preferably, a combination of mutations consisting of M428L/N434S or T256D/T307Q (DQ) or T256D/T307W (DW) or M252Y/T256D (YD) or T307Q/Q311V/A383V or T256D/H286D/T307R/Q311V/A378A or L309D/Q311H/N434S (DHS) or M252Y/S254T/T256E (MST-YTE) is introduced into the IgG1 Fc domain. These aspects may be combined with mutations above to incorporate the selected functionalities.

In a fifth aspect, there is provided a cell comprising an antibody according to any previous aspect of the invention. Preferably, the antibody is in a complex bound to tau protein. The cell may be a neuronal cell. Preferably, the antibody is within the cytoplasmic compartment of the cell.

In a sixth aspect, the invention provides a method for neutralising a target in a cell, comprising administering to the cell an antibody specific for the target, said antibody being modified to increase binding to Trim21 in comparison to an unmodified antibody, by introducing a T256P mutation and/or by modification of the antibody hinge region, such as by replacement of the hinge region with an IgG3 hinge region.

TRIM21 is effective in mediating degradation of proteins or other macromolecules, as well as viruses, which are taken up by the cell, if the antibody is taken up into the cell along with the target. We show herein that antibodies are not only translocated into the cell with the target, but are also effective in neutralising the target once they reach the cellular cytoplasm.

The target can be any molecule which can transit into a cell when attached to an antibody, for example a target selected from the group consisting of viruses, protein aggregates, tau, alpha-synuclein, TDP43 and SOD1.

In one example, the target is a misfolded or aggregated form of a protein, such as tau, TDP-43 or alpha-synuclein, that possesses the ability to template further aggregation of the same protein in the cytoplasm.

The antibody is preferably administered extracellularly and allowed to bind to the target, such that it is introduced into the cell in association with the target. Thus, intravenous administration, together with other methods for extracellular administration, is possible.

Preferably, the antibody is an antibody according to any one of the former aspects of the present invention.

In a seventh aspect, the invention provides a cell comprising with its cytoplasm an antibody according to any preceding aspect, which is bound to tau. The cell is preferably a neuronal cell.

According to an eighth aspect, there is provided a method for treating or preventing a tau pathology in a subject, comprising administering to the subject an anti-tau antibody according to any one of the second to the fourth aspects of the present invention, as an extracellular preparation. In some embodiments, the administration is made intravenously.

In some embodiments, the antibody is an antibody in which the ability to bind to FcγR and/or complement (C1q) has been reduced or eliminated.

In some embodiments, FcγR binding is reduced as set forth in respect of the preceding aspects of the invention.

In some embodiments, antibody recycling via FcRn is enhanced. Methods of enhancing FcRn recycling are set forth above, in the preceding aspects of the invention.

### BRIEF DESCRIPTION OF THE FIGURES

FIG. 1. Mechanisms of antibody protection in neuronal culture.
a) Schematic of the tau entry assay. Assemblies of recombinant tau-HiBiT are added to the primary neurons expressing cytosolic LgBiT. Entry can be quantified in real time using cell-penetrant luciferase substrate. b) Levels of phosphorylated tau-HiBiT or c) tau-HiBiT entry to primary mouse neuron cytosol following incubation with control Ab (anti-adenovirus 9C12) or tau-binding Abs A0024 and polyclonal mouse anti-tau, mIgG-T and anti-phospho-tau Ab AP422. d) Diagram of organotypic slice culture (OHSC) model of seeded tau aggregation. Tau assemblies are pre-treated with Abs and provided to hippocampal slices prepared from P301S Tau-Tg animals on DIV7. On DIV10 media is changed. OHSCs are fixed for immunofluorescence analysis of tau pathology (AT8) on DIV28 or examined for levels of tau seeding in HEK293 P301S tau-venus reporter cells. e, f) Levels of AT8 pathology in OHSCs following challenge with tau assemblies or phospho-tau assemblies pre-treated with the indicated Ab. g) Levels of AT8 staining in OSHCs derived from T21^{+/+} and T21^{-/-} backgrounds with and without tau assemblies at the indicated time in vitro. h) Levels of AT8 staining in T21^{+/+} and T21^{-/-} OHSCs treated with tau assemblies and the indicated Abs. i) Representative immunofluorescence images for AT8-reactive tau structures in OHSCs from T21^{+/+} and T21^{-/-} backgrounds challenged with tau assemblies that were untreated or incubated with the indicated Ab. j) Levels of seeding observed in extracts prepared from OHSCs treated with the indicated tau assemblies and Abs. k) Levels of AT8 staining in OHSCs treated with tau assemblies that were incubated with the indicated recombinant Abs. Ragweed, anti-ragweed pollen control; AP422_{WT}, mouse IgG2a; AP422_{PGLALA}, mouse IgG2a with the PGLALA mutations that prevent FcγR interaction. Error bars, sem. All OHSC data represent images from multiple slices from N=6 mice. c, e, f, j and k, one-way ANOVA with Tukey's correction for multiple comparisons. b, unpaired t-test. g, and h, two-way ANOVA.**, P<0.01; ***, P<0.001; **** P<0.0001.

FIG. 2. T21 protects against incipient tau pathology in P301S-Tg mice.
a) Levels of TRIM21, synaptic marker PSD-95, IFN-stimulated gene STAT-1 and loading control CypB in human iPSC derived neurons in the presence and absence of IFNα. b) Levels of adenovirus type 5 infection in human iPSC derived neurons in the absence of Ab or in the presence of recombinant humanised anti-AdV 9C12. Mouse-human chimeric 9C12 versions were made with a wildtype human IgG1 Fc and with the H433A substitution which prevents interaction with TRIM21. c) Western blots of lysate and sarkosyl insoluble (SI) fractions prepared from the lumbar spinal column of P301S-Tg mice at postnatal day 20, 50 and 80. Lanes represent individual animals. d) Quantification of tau is SI fractions using antibodies AT100 and HT7, normalised to GAPDH. e) Levels of seeding in HEK293 P301S tau-venus cells treated with the same SI fractions, or with SI fractions from wildtype mice. f) Western blot of total and sarkosyl insoluble fractions of spines from P301S-Tg mice treated with mock i.p. injection (PBS), control antibody (9C12) or anti-tau (AP422) between ages 20-80 days. g) Quantification of AT100 levels normalised to GAPDH from f). h) Levels of seed competent tau present in spine lysates derived from mice treated with the indicated Ab. Error bars, sem. g and h, one-way ANOVA; *** P<0.001; **** P<0.0001.

FIG. 3. Long-term T21-dependent protection against tau and evidence of T21 activity in human neurons.
a,b) Western blot of sarkosyl insoluble (SI) and input fractions from mice that were untreated or treated with polyclonal anti-tau mIgG-T or AP422 for 17 weeks by intraperitoneal injection. Mice were either T21^{+/+} (a) or T21^{-/-} (b). nd, lane not determined due to insufficient sample for analysis. c,d) Quantification of blots for c) T21^{+/+} and d) T21^{-/-} animals. All band intensities were normalised to input GAPDH. Error bars, sem. One-way ANOVA; * P<0.05; ** P<0.01 *** P<0.001.

FIG. 4. Antibodies can exert intracellular protection against seeded tau aggregation.
HEK293 cells expressing P301S tau-venus were electroporated with control antibody 9C12 or anti-tau antibodies AP422, BR134 and AT8. Each of the anti-tau antibodies reduced seeded aggregation after cells were challenged with recombinant tau assemblies which were introduced to the cells with lipofectamine.

FIG. 5. Deglycosylation does not prevent intracellular function of antibodies.
(A) HEK293T cells transfected with a NF-κB driven luciferase construct treated with human adenovirus type 5 (AdV) incubated with IgG treated with BSA, Neuraminidase (Neura), PNGase F or the full deglycosylation mix (DG mix). (B) As (A), but in neutralisation assay. (C) Denaturing SDS-PAGE of antibodies from (A). (D) As (A), but with IgM. (E) As (B), but with IgM. (F) SDS-PAGE of IgM from (D). (G) As (A), but with IgA. Results shown as mean of triplicate data, with standard error of the mean; presented in (A, D, G) as fold change over PBS treated cells, and (B, E) as normalised percentage of GFP positive cells.

FIG. 6. Characterisation of antibodies. a) Dot blot using phospho-tau antibody AP422 or anti-adenovirus antibody 9C12 against immobilised recombinant (rec) tau, which is not phosphorylated, and sarkosyl insoluble (SI) tau prepared from P301S tau transgenic mice, which is hyperphosphorylated. b) Dot blot showing levels of AP422 reactivity against SI tau prepared from brain tissue from histologically confirmed Alzheimer's disease (AD), corticobasal degeneration (CBD) and progressive supranuclear palsy (PSP) or an individual with no observable tau pathology (Con). A dilution series of SI fraction corresponding to equal volumes of brain tissue was loaded for each patient. c) Graph of relative reactivity of AP422 to SI preparations, as measured by dot blot in (b). d) Dot blot showing binding by tau antibodies BR134 and Dako A0024 to recombinant and SI tau as in (a). e) Epitope mapping of mIgG-T using overlapping peptides of ON4R tau. Amino acid refers to start site of 15mer peptides.

FIG. 7. Organotypic slice cultures. a) Tiled fluorescence microscope images of OHSCs prepared from P301S Tau-Tg T21^{+/+} and P301S Tau-Tg T21/mouse strains immunostained for Map2 (neuronal marker), Iba1 (microglial marker) and Gfap (astrocyte marker). b) Western blot demonstrating expression of Trim21 in OHSCs prepared from P301S Tau-Tg mice that were T21^{+/+} or T214. CypB, cyclophilin B loading control. c) Levels of AT8 reactivity following treatment with recombinant tau assemblies that were untreated or treated with kinases. Error bars, sem. Kruskall-Wallace test with multiple comparisons *, P<0.05; **** P<0.0001.

FIG. 8. Protection conferred by BR134 is T21-dependent. a) Levels of AT8 reactivity in OHSCs following treatment with tau assemblies in the absence of Abs, or after pre-incubation with control Ab 9C12 or C-terminal targeting Ab BR134. b) Levels of tau seeds present within OHSCs following treatment with indicated tau and Ab complexes. Levels were determined by applying lysate to HEK293 cells expressing P301S tau-venus.

FIG. 9. ELISA measurement of AP422 IgG2a binding to mouse FcγRs. Recombinant AP422 was made as mouse IgG2a and expressed as wildtype or as an Fc-engineered version containing the PGLALA substitutions. Titrated amounts of the antibodies were coated in ELISA wells followed by addition of recombinant soluble forms of the following receptors that were site-specifically biotinylated. a) mFcγRI (Fcgr1, CD64); b) mFcγRIIb (Fcgr2, CD32); c) mFcγRIII (Fcgr3, CD16); d) mFcγRIV (Fcgr4, CD16.2); c); d). Bound receptors were detected using alkaline phosphate-conjugated streptavidin.

FIG. 10. Neutralisation of adenovirus by modified antibodies a) Western blot of TRIM21 in unmodified (WT) HEK293T or HEK293T cells treated with CRISPR/Cas9 targeting TRIM21 (TRIM21 KO). Cells were treated with IFNα or IFNβ at 5000U/ml overnight, which upregulates TRIM21, to confirm that levels of TRIM21 remain undetectable. b) Levels of neutralisation of AdV5-GFP treated with indicated concentrations of 9C12 antibodies after 24 hrs. Mean±SEM of triplicates from three independent experiments. c) Binding of recombinant 9C12 mutants to AdV-5 hexon (1 μg/ml) in ELISA. d) Fluorescence anisotropy with 5 nM human TRIM21 PRYSPRY labelled with Alexa Fluor 488 mixed with indicated concentration of 9C12 antibodies. Binding curve fitted using non-linear regression analysis e) Binding of recombinant 9C12 antibodies to human FcγR1 via HTRF. Dissociation constant derived using non-linear regression. f) Denaturing SDS-PAGE stained with Coomassie blue showing migration of recombinant 9C12 mutants. g) Neutralisation of AdV5-GFP in complex with 6 ng/ml of recombinant 9C12 antibodies after 24 hrs in HEK293T WT or TRIM21 KO conditions. Mean±SD of triplicates from four independent experiments. h) Neutralisation of AdV5-GFP in complex with 20 ng/ml of recombinant 9C12 antibodies after 24 hrs. Mean±SD of triplicates from two independent experiments. i) TRIM21-mediated neutralisation of AdV5-GFP in complex with indicated concentrations of 9C12 antibodies after 24 hrs. Mean±SEM of triplicates from one experiment. Fold neutralisation in infection experiments calculated as level of infection in virus only condition divided by level of infection by antibody-treated virus. All statistical analyses are one-way ANOVA with Šidák correction for multiple comparisons, *** P<0.001; **** P<0.0001; ns, not significant.

FIG. 11. In vivo protection conferred by anti-tau antibodies of IgG classes with different affinity to FcγR
a) ELISA measurement of recombinant AP422-wt msIgG1, AP422-wt msigG2a, and AP422-N297A L234A L235A msIgG2a binding to mouse FcγRI. Antibodies were used to coat ELISA wells followed by addition of recombinant soluble forms of biotinylated mFcγRI (Fcgr1, CD64). Bound receptors were detected using alkaline phosphate-conjugated streptavidin. b) Fluorescence anisotropy of 5 nM Alexa488-labelled mouse T21 PRYSPRY domain in the presence of indicated concentration of msIgG1 AP422 or msIgG2a AP422. c) Dot blot against immobilised recombinant tau assemblies (rec), P301S Tau-Tg mouse brain sarkosyl insoluble tau (SI) or recombinant tau assemblies that were untreated or treated with ERK2 kinase. Membranes were probed using either HT7 (total tau); recombinant AP422 expressed as either mouse IgG1 or mouse IgG2a; the tau repeat region-specific Ab, DAKO A0024; or commercial rabbit phospho-tau specific antibody, anti-pS422. d) Percent of HEK293 tau-venus cells seeded after challenge with 0.25 nM phosphorylated P301S tau assemblies that were mixed with 1.25 nM of indicated antibodies for 1 h before addition to culture supernatant. e) Immunoblot analysis of sarkosyl insoluble fractions of spines from treated mice at 30 mg/kg by weekly ip injection for 9 weeks. Insoluble phospho-tau was detected using antibody AT100. Levels of HSP60 in input serve as loading control. Each lane represents an individual mouse. f) Quantification of sarkosyl insoluble AT100 levels normalised to HSP60 from e). g) Levels of seed competent tau present in spine sarkosyl insoluble fractions derived from mice treated with the indicated Ab in the HEK293 tau-venus system, points represent average seeding in images broken down by individual mouse. Mean with sem. f), one-way ANOVA with Dunnett's multiple comparison test. g) nested one-way ANOVA; * P<0.05; ** P<0.01; *** P<0.001.

FIG. 12. Neutralisation of Tau seeding by modified antibodies
a) Dot blot against immobilised recombinant tau assemblies (rec), P301S Tau-Tg mouse brain sarkosyl insoluble tau (SI) or recombinant tau assemblies that were untreated or treated with ERK2 kinase. Membranes were probed using either recombinant AP422 antibodies expressed as human-mouse chimeras with IgG1 Fc region, commercial rabbit phospho-tau specific antibodies, anti-pS422 and anti-pS396, or the tau repeat region-specific antibody, DAKO A0024. b) and f) Binding of recombinant AP422 antibodies to human FcγR1 via HTRF. Mean±SD of three repeats; curves were fitted using non-linear regression. c) and g) Human iPSC-derived microglia were incubated with 0.05 nM sarkosyl insoluble tau extracted from passaged HEK293 cells preincubated with 250 nM of the indicated recombinant mouse-human IgG1 Fc chimeric AP422 antibody or PBS control for 24 hours, followed by media collection and measurement of TNFα by ELISA. Mean±SD of three repeats; one-way ANOVA with Sidák correction for multiple comparisons. d) Denaturing SDS-PAGE gel stained with Coomassie blue showing migration of indicated recombinant AP422 antibodies. e) Binding of recombinant AP422 antibodies to human FcRn via HTRF. Mean±SD of three repeats. h.) ELISA measurement of interaction between human FcRn (1 μg/ml) and indicated concentration of recombinant AP422 antibodies at either pH 6 or pH 7.4. i) and j) Fluorescence anisotropy of 5 nM human TRIM21 PRYSPRY labelled with Alexa Fluor 488 in the presence of indicated concentration of AP422 antibodies. Mean±SD of three repeats. Binding curve fitted using non-linear regression analysis. k) Levels of seeding in HEK293 cells expressing P301S tau-venus 72 hrs after treatment with 0.25 nM phosphorylated, recombinant P301S tau assemblies preincubated with 1.25 nM AP422 antibodies. Mean±SD of triplicates from two independent experiments; one-way ANOVA with Dunnett's correction for multiple comparisons. ** P<0.01; *** P<0.001; **** P<0.0001; ns, not significant.

FIG. 13. Tau assemblies enter neurons in complex with anti-tau antibodies to contact TRIM21
a) Confocal immunofluorescence microscope images of mouse primary neurons expressing mCherry-T21 treated with tau assemblies in complex with tau C-terminus specific rabbit polyclonal Ab, BR134. Arrows indicate intracellular Ab: tau assembly complexes, the majority of which were found to colocalise with T21. Control images demonstrating the absence of mCh-T21 foci at site of intracellular tau assemblies when antibody is absent. Scale bar 25 μm, inset scale bar 10 μm. b) Fluorescence anisotropy of Alexa488-labelled mouse T21 PRYSPRY domain in the presence of indicated concentration of BR134. c) Primary neurons prepared from T21^{-/-} mice were treated with indicated amounts of chimeric AAV1/2 particles encoding mCherry-tagged mouse Trim21 under the hSyn promoter. Immunoblot for TRIM21 and loading control GAPDH. vg, viral genomes. d) Uptake of tau assemblies is unaffected by BR134 8 h after addition to neurons. e) Percent of intracellular tau assemblies positive for mCh-T21 puncta in the presence or absence of BR134. f) Number of intracellular tau puncta that colocalise with T21 in the presence or absence of BR134. ns, not significant, **, P<0.01 Mann-Whitney U-test.

### DETAILED DESCRIPTION OF THE INVENTION

Unless otherwise stated, all technical and scientific terms used herein have the same meanings as commonly understood by one of ordinary skill in the art to which this invention belongs. Any methods and materials similar or equivalent to those described herein can be used in the practice or testing of the present invention. Methods, devices, and materials suitable for such uses are now described. All publications cited herein are incorporated herein by reference in their entirety for the purpose of describing and disclosing the methodologies, reagents, and tools reported in the publications that might be used in connection with the invention.

The practice of the present invention employs, unless otherwise indicated, conventional methods of chemistry, biochemistry, molecular biology, cell biology, genetics, immunology and pharmacology, known to those of skill of the art. Such techniques are explained fully in the literature. See, e. g., Gennaro, A. R., ed. (1990) Remington's Pharmaceutical Sciences, ¹8th ed., Mack Publishing Co.; Hardman, J. G., Limbird, L. E., and Gilman, A. G., eds. (2001) The Pharmacological Basis of Therapeutics, 10th ed., McGraw-Hill Co.; Colowick, S. et al., eds., Methods In Enzymology, Academic Press, Inc.; Weir, D. M., and Blackwell, C. C., eds. (1986) Handbook of Experimental Immunology, Vols. I-IV, Blackwell Scientific Publications; Maniatis, T. et al., eds. (1989) Molecular Cloning: A Laboratory Manual 2nd edition, Vols. I-III, Cold Spring Harbor Laboratory Press; Ausubel, F. M. et al., eds. (1999) Short Protocols in Molecular Biology, 4th edition, John Wiley & Sons; Ream et al., eds. (1998) Molecular Biology Techniques: An Intensive Laboratory Course, Academic Press; Newton, C. R., and Graham, A., eds. (1997) PCR (Introduction to Biotechniques Series)· 2nd ed., Springer Verlag.

In the context of the present invention, administration is performed by standard techniques of cell culture, depending on the reagent, compound or gene construct to be administered. For instance, administration may take place by addition to a cell culture medium, introduction into cells by precipitation with calcium phosphate, by electroporation, by viral transduction or by other means. If the method of the invention employs a non-human mammal as the test system, the mammal may be transgenic and express the necessary reagents in its endogenous cells.

Extracellular administration is the administration of an agent, composition or compound to an extracellular environment, such as the cell medium in a cellular culture, intravenous or intraperitoneal administration to an organism, or the like. Extracellular administration excludes techniques which are designed to transport the administered substance into the cell by non-natural processes, including microinjection, electroporation, transfection, and the like.

An antigen, in the context of the present invention, is a molecule which can be recognised by a ligand and which possesses an epitope recognised by said ligand, such as the binding site for an antibody. Typically, an antigen is an antigenic determinant of a target, especially an antigenic determinant of tau or tau aggregates or assemblies, and is exposed to binding by ligands such as antibodies under physiological conditions. Preferred antigens comprise epitopes targeted by known anti-tau antibodies.

A ligand which binds directly to an antigen is a ligand which is capable of binding specifically to an antigen under physiological conditions. As used herein, the term “ligand” can refer to either part of a specific binding pair; for instance, it can refer to the antibody or the antigen in an antibody-antigen pair. Antibodies are preferred ligands, and may be complete antibodies or antibody fragments as are known in the art, comprising for example IgG, IgA, IgM, IgE, IgD, F (‘ab’)₂, Fab, Fv, scFv, dAb, V_{HH}, IgNAR, a modified TCR, and multivalent combinations thereof. IgG antibodies are preferred, and may comprise IgG1, IgG2, IgG3 and/or IgG4. Ligands may also be binding molecules based on alternative non-immunoglobulin scaffolds, peptide aptamers, nucleic acid aptamers, structured polypeptides comprising polypeptide loops subtended on a non-peptide backbone, natural receptors or domains thereof, small molecules and other agents capable of specific binding.

Where the ligand is an antibody, it preferably retains the Fc domain, which is responsible for binding to TRIM21. Where the ligand is an antibody fragment, an Fc domain or other TRIM21 binding domain may be attached.

The term “immunoglobulin” refers to a family of polypeptides which retain the immunoglobulin fold characteristic of antibody molecules, which contains two beta sheets and, usually, a conserved disulphide bond. Members of the immunoglobulin superfamily are involved in many aspects of cellular and non-cellular interactions in vivo, including widespread roles in the immune system (for example, antibodies, T-cell receptor molecules and the like), involvement in cell adhesion (for example the ICAM molecules) and intracellular signalling (for example, receptor molecules, such as the PDGF receptor). The present invention is applicable to all immunoglobulin superfamily molecules which possess binding domains. Preferably, the present invention relates to antibodies.

The variable domains of the heavy and light chains of immunoglobulins, and the equivalents in other proteins such as the alpha and beta chains of T-cell receptors, are responsible for determining antigen binding specificity. V_{H} and V_{L} domains are capable of binding antigen independently, as in V_{H} and V_{L} dAbs. References to V_{H} and V_{L} domains include modified versions of V_{H} and V_{L} domains, whether synthetic or naturally occurring. For example, naturally occurring V_{H} variants include camelid V_{HH} domains, and the heavy chain immunoglobulins IgNAR of cartilaginous fish.

An “Fc” or “Fc domain” or “Fc region”, as used herein is the constant region of an antibody excluding the first constant region immunoglobulin domain. Thus, Fc refers to the last two constant region immunoglobulin domains of IgA, IgD, and IgG, and the last three constant region immunoglobulin domains of IgE and IgM, and the flexible hinge N-terminal to these domains. For IgA and IgM, Fc may include the J chain. For IgG, Fc comprises immunoglobulin domains C_{H}2 and C_{H}3 and the hinge between C_{H}1 and C_{H}2. Although the boundaries of the Fc region may vary, the human IgG heavy chain Fc region is usually defined to comprise residues C226 or P230 to its carboxyl-terminus.

Ligands according to the present invention are capable of being bound by TRIM21 in the cell cytoplasm. For example, they retain or comprise an Fc domain, such as an IgG1 Fc domain, which is bound by TRIM21.

Ligands according to the present invention bind to tau seeds, assemblies or aggregates before they enter the cell cytoplasm. We show herein that upon cellular uptake these antibodies remain bound to tau and target it for degradation in the proteasome via the E3 ubiquitin ligase activity of TRIM21.

Tau is a microtubule associate protein encoded by the MAPT gene. It is responsible for assembly of microtubules. Tau is believed to be a causative agent in neurodegenerative disease, forming hyperphosphorylated fibrillar assemblies in neuronal cytoplasm; tau pathology is believed to spread in a prion-like manner during neurodegenerative disease. As used herein, a reference to “tau” is a reference to any form of the tau protein, including normal cellular tau as well as tau assemblies, fibrils, aggregates and other abnormal conformations. It includes post-translationally modified versions of tau including phosphorylated, acetylated, glycosylated, ubiqutinated and otherwise-modified variants. It also includes mutant forms of tau, especially mutant forms which are associated with genetically transmitted predisposition to neurodegenerative diseases.

TRIM21 is a member to the tripartite motif-containing family of proteins, the sequence of which can be accessed as P19474 in the UniProt database. Reference herein to TRIM21 is typically a reference to human TRIM21.

Antibody modification, as referred to herein, may be carried out by effecting point mutations as described. Residue numbers typically refer to the Eu antibody sequence standard. By “amino acid modification” herein is meant an amino acid substitution, insertion, and/or deletion in a polypeptide sequence. By “amino acid substitution” or “substitution” herein is meant the replacement of an amino acid at a particular position in a parent polypeptide sequence with another amino acid. For example, the substitution L234A refers to a variant antibody Fc domain in which the leucine at position 234 is replaced with alanine.

An antibody which is adapted for intracellular activity is an antibody in which a specific modification has been made to enhance the intracellular activity of the ligand, for instance to enhance interaction with TRIM21.

Other modifications may be made. For example, reduction in FcγR binding reduces the inflammatory effect of antibodies, particularly in neuronal immunotherapy.

By “Fc γ receptor” or “FcγR” as used herein is meant any member of the family of proteins that bind the IgG antibody Fc region and are substantially encoded by the FcγR genes. In humans this family includes but is not limited to FcγR1 (CD64), including isoforms FcγR1a, FcγR1b, and FcγR1c; FcγRII (CD32), including isoforms FcγRIIa (including allotypes H131 and R131), FcγRIIb (including FcγRIIb-1 and FcγRIIb-2), and FcγRIIc and FcγRIII (CD16), including isoforms FcγRIIIa (including allotypes V158 and F158) and FcγRIIIb (including allotypes FcγRIIIb-NA1 and FcγRIIb-NA2) (Jefferis et al, 2002, Immunol Lett 82:57-65,), as well as any undiscovered human FcγRs or FcγR isoforms or allotypes. Mouse FcγRs include but are not limited to FcγRI (CD64), FcγRII (CD32), FcγRII (CD16), and FcγRIII-2 (CD16-2).

The present invention provides isolated nucleic acids encoding the ligands described herein. The present invention provides vectors comprising the nucleic acids, optionally, operably linked to control sequences. The present invention provides host cells containing the vectors, and methods for producing and optionally recovering the ligands.

The present invention provides novel ligands, including antibodies, Fc fusions with binding domains, and non-antibody ligands that bind tau and TRIM21. The ligands are useful in a therapeutic product. In certain embodiments, the Fc polypeptides of the invention are antibodies.

The present invention provides compositions comprising ligands, such as antibodies, described herein, and a physiologically or pharmaceutically acceptable carrier or diluent.

### 1. Ligands

Any ligand which can bind to a tau protein and to TRIM21 under physiological conditions, and be internalized by a cell either alone or in complex with tau assemblies, is suitable for use in the present invention. The natural immune system uses antibodies as ligands, and antibodies or antibody fragments are ideal for use in the present invention. Other possibilities include binding domains from other receptors, as well as engineered peptides, nucleic acids and other small molecules.

1a. Antibodies

References herein to tau-specific antibodies, antigen- or peptide-binding antibodies and antibodies specific for an antigen are coterminous and refer to antibodies, or binding fragments derived from antibodies, which bind to antigens and especially tau in a specific manner and substantially do not cross-react with other molecules present in the circulation or the tissues.

An “antibody” as used herein includes but is not limited to, polyclonal, monoclonal, recombinant, chimeric, complementarity determining region (CDR)-grafted, single chain, bi-specific, Fab fragments and fragments produced by a Fab expression library. Such fragments include fragments of whole antibodies which retain their binding activity for the desired antigen, Fv, F (‘ab’), F (‘ab’)₂ fragments, and F (v) or V_{H} antibody fragments as well as fusion proteins and other synthetic proteins which comprise the antigen-binding site of the antibody. Furthermore, the antibodies and fragments thereof may be human or humanized antibodies, as described in further detail below.

Antibodies and fragments also encompass antibody variants and fragments thereof. Variants include peptides and polypeptides comprising one or more amino acid sequence substitutions, deletions, and/or additions that have the same or substantially the same affinity and specificity of epitope binding as the antigen-specific antibody or fragments thereof.

The deletions, insertions or substitutions of amino acid residues may produce a silent change and result in a functionally equivalent substance. Deliberate amino acid substitutions may be made on the basis of similarity in polarity, charge, solubility, hydrophobicity, hydrophilicity, and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine. Conservative substitutions may be made, for example according to the Table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

Homologous substitution (substitution and replacement are both used herein to mean the interchange of an existing amino acid residue, with an alternative residue) may occur i.e. like-for-like substitution such as basic for basic, acidic for acidic, polar for polar etc. Non-homologous substitution may also occur i.e. from one class of residue to another.

Thus, variants may include peptides and polypeptides comprising one or more amino acid sequence substitutions, deletions, and/or additions to the antigen specific antibodies and fragments thereof wherein such substitutions, deletions and/or additions do not cause substantial changes in affinity and specificity of epitope binding. Variants of the antibodies or fragments thereof may have changes in light and/or heavy chain amino acid sequences that are naturally occurring or are introduced by in vitro engineering of native sequences using recombinant DNA techniques. Naturally occurring variants include “somatic” variants which are generated in vivo in the corresponding germ line nucleotide sequences during the generation of an antibody response to a foreign antigen.

Variants of antibodies and binding fragments may also be prepared by mutagenesis techniques. For example, amino acid changes may be introduced at random throughout an antibody coding region and the resulting variants may be screened for binding affinity for the target antigen, or for another property. Alternatively, amino acid changes may be introduced into selected regions of the antibody, such as in the light and/or heavy chain CDRs, and/or in the framework regions, and the resulting antibodies may be screened for binding to the target antigen or some other activity. Amino acid changes encompass one or more amino acid substitutions in a CDR, ranging from a single amino acid difference to the introduction of multiple permutations of amino acids within a given CDR. Also encompassed are variants generated by insertion of amino acids to increase the size of a CDR.

The antigen-binding antibodies and fragments thereof may be humanized or human engineered antibodies. As used herein, “a humanized antibody”, or antigen binding fragment thereof, is a recombinant polypeptide that comprises a portion of an antigen binding site from a non-human antibody and a portion of the framework and/or constant regions of a human antibody. A human engineered antibody or antibody fragment is a non-human (e.g., mouse) antibody that has been engineered by modifying (e.g., deleting, inserting, or substituting) amino acids at specific positions so as to reduce or eliminate any detectable immunogenicity of the modified antibody in a human.

Humanized antibodies include chimeric antibodies and CDR-grafted antibodies. Chimeric antibodies are antibodies that include a non-human antibody variable region linked to a human constant region. Thus, in chimeric antibodies, the variable region is mostly non-human, and the constant region is human. Chimeric antibodies and methods for making them are described in, for example, Proc. Natl. Acad. Sci. USA, 81:6841-6855 (1984). Although, they can be less immunogenic than a mouse monoclonal antibody, administrations of chimeric antibodies have been associated with human immune responses (HAMA) to the non-human portion of the antibodies.

CDR-grafted antibodies are antibodies that include the CDRs from a non-human “donor” antibody linked to the framework region from a human “recipient” antibody. Methods that can be used to produce humanized antibodies also are described in, for example, U.S. Pat. Nos. 5,721,367 and 6, 180,377.

“Veneered antibodies” are non-human or humanized (e.g., chimeric or CDR-grafted antibodies) antibodies that have been engineered to replace certain solvent-exposed amino acid residues so as to reduce their immunogenicity or enhance their function. Veneering of a chimeric antibody may comprise identifying solvent-exposed residues in the non-human framework region of a chimeric antibody and replacing at least one of them with the corresponding surface residues from a human framework region. Veneering can be accomplished by any suitable engineering technique.

Further details on antibodies, humanized antibodies, human engineered antibodies, and methods for their preparation can be found in Antibody Engineering, Springer, New York, NY, 2001.

Examples of humanized or human engineered antibodies are IgG, IgM, IgE, IgA, and IgD antibodies. The antibodies may be of any class (IgG, IgA, IgM, IgE, IgD, etc.) or isotype and can comprise a kappa or lambda light chain. For example, a human antibody may comprise an IgG heavy chain or defined fragment, such as at least one of isotypes, IgG1, IgG2, IgG3 or IgG4. As a further example, the antibodies or fragments thereof can comprise an IgG1 heavy chain and a kappa or lambda light chain.

The antigen specific antibodies and fragments thereof may be human antibodies-such as antibodies which bind the antigen and are encoded by nucleic acid sequences which may be naturally occurring somatic variants of human germline immunoglobulin nucleic acid sequence, and fragments, synthetic variants, derivatives and fusions thereof. Such antibodies may be produced by any method known in the art, such as through the use of transgenic mammals (such as transgenic mice) in which the native immunoglobulins have been replaced with human V-genes in the mammal chromosome.

Human antibodies to target a desired antigen can also be produced using transgenic animals that have no endogenous immunoglobulin production and are engineered to contain human immunoglobulin loci, as described in WO 98/24893 and WO 91/00906.

Human antibodies may also be generated through the in vitro screening of antibody display libraries (J. Mol. Biol. (1991) 227:381). Various antibody-containing phage display libraries have been described and may be readily prepared. Libraries may contain a diversity of human antibody sequences, such as human Fab, Fv, and scFv fragments, that may be screened against an appropriate target. Phage display libraries may comprise peptides or proteins other than antibodies which may be screened to identify agents capable of selective binding to the desired antigen.

Phage-display processes mimic immune selection through the display of antibody repertoires on the surface of filamentous bacteriophage, and subsequent selection of phage by their binding to an antigen of choice. One such method is described in WO 99/10494. Antigen-specific antibodies can be isolated by screening of a recombinant combinatorial antibody library, preferably a scFv phage display library, prepared using human V_{L} and V_{H} CDNAs prepared from mRNA derived from human lymphocytes. Methodologies for preparing and screening such libraries are known in the art. There are commercially available kits for generating phage display libraries.

As used herein, the term “antibody fragments” refers to portions of an intact full length antibody-such as an antigen binding or variable region of the intact antibody. Examples of antibody fragments include Fab, F′ab′, F (‘ab’)₂, and Fv fragments; diabodies; linear antibodies; single-chain antibody molecules (e.g., scFv); multispecific antibody fragments such as bispecific, trispecific, and multispecific antibodies (e.g., diabodies, triabodies, tetrabodies); binding-domain immunoglobulin fusion proteins; camelized antibodies; minibodies; chelating recombinant antibodies; tribodies or bibodies; intrabodies; nanobodies; small modular immunopharmaceuticals (SMIP), V_{HH} containing antibodies; and any other polypeptides formed from antibody fragments.

The antigen binding antibodies and fragments encompass single-chain antibody fragments (scFv) that bind to the desired antigen. An scFv comprises an antibody heavy chain variable region (V_{H}) operably linked to an antibody light chain variable region (V_{L}) wherein the heavy chain variable region and the light chain variable region, together or individually, form a binding site that binds to the antigen. An scFv may comprise a V_{H} region at the amino-terminal end and a V_{L} region at the carboxy-terminal end. Alternatively, scFv may comprise a V_{L} region at the amino-terminal end and a V_{H} region at the carboxy-terminal end. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv). An scFv may optionally further comprise a polypeptide linker between the heavy chain variable region and the light chain variable region.

The antigen binding antibodies and fragments thereof also encompass immunoadhesins. One or more CDRs may be incorporated into a molecule either covalently or noncovalently to make it an immunoadhesin. An immunoadhesin may incorporate the CDR(s) as part of a larger polypeptide chain, may covalently link the CDR(s) to another polypeptide chain, or may incorporate the CDR(s) noncovalently. The CDRs permit the immunoadhesin to specifically bind to the desired antigen.

The antigen binding antibodies and fragments thereof also encompass antibody mimics comprising one or more antigen binding portions built on an organic or molecular scaffold (such as a protein or carbohydrate scaffold). Proteins having relatively defined three-dimensional structures, commonly referred to as protein scaffolds, may be used as reagents for the design of antibody mimics. These scaffolds typically contain one or more regions which are amenable to specific or random sequence variation, and such sequence randomization is often carried out to produce libraries of proteins from which desired products may be selected. For example, an antibody mimic can comprise a chimeric non-immunoglobulin binding polypeptide having an immunoglobulin-like domain containing scaffold having two or more solvent exposed loops containing a different CDR from a parent antibody inserted into each of the loops and exhibiting selective binding activity toward a ligand bound by the parent antibody. Non-immunoglobulin protein scaffolds have been proposed for obtaining proteins with novel binding properties.

Antigen specific antibodies or antibody fragments thereof typically bind to the desired antigen with high affinity (e.g., as determined with BIAcore), such as for example with an equilibrium binding dissociation constant (K_{D}) for the antigen of about 15 nM or less, 10 nM or less, about 5 nM or less, about 1 nM or less, about 500 pM or less, about 250 pM or less, about 100 pM or less, about 50 pM or less, or about 25 pM or less, about 10 pM or less, about 5 pM or less, about 3 pM or less about 1 pM or less, about 0.75 pM or less, or about 0.5 pM or less.

### 1b Peptide Ligands

Peptides, such as peptide aptamers, can be selected from peptide libraries by screening procedures. In practice, any vector system suitable for expressing short nucleic acid sequences in a eukaryotic cell can be used to express libraries of peptides. In a preferred embodiment, high-titer retroviral packaging systems can be used to produce peptide aptamer libraries. Various vectors, as well as amphotropic and ecotropic packaging cell lines, exist that can be used for production of high titers of retroviruses that infect mouse or human cells. These delivery and expression systems can be readily adapted for efficient infection of any mammalian cell type, and can be used to infect tens of millions of cells in one experiment. Aptamer libraries comprising nucleic acid sequences encoding random combinations of a small number of amino acid residues, e.g., 5, 6, 7, 8, 9, 10 or more, but preferably less than 100, more preferably less than 50, and most preferably less than 20, can be expressed in retrovirally infected cells as free entities, or depending on the target of a given screen, as fusions to a heterologous protein, such as a protein that can act as a specific protein scaffold (for promoting, e.g., expressibility, intracellular or intracellular localization, stability, secretability, isolatablitiy, or detectability of the peptide aptamer. Libraries of random peptide aptamers when composed of, for example 7 amino acids, encode molecules large enough to represent significant and specific structural information, and with 107 or more possible combinations is within a range of cell numbers that can be tested.

Preferably, the aptamers are generated using sequence information from the target antigen.

In identifying an aptamer, for example, a population of cells is infected with a gene construct expressing members of an aptamer library, and the ability of aptamers to bind to an antigen is assessed, for instance on a BIAcore platform. Coding sequences of aptamers selected in the first round of screening can be amplified by PCR, re-cloned, and re-introduced into naïve cells. Selection using the same or a different system can then be repeated in order to validate individual aptamers within the original pool. Aptamer coding sequences within cells identified in subsequent rounds of selection can be iteratively amplified and subcloned and the sequences of active aptamers can then be determined by DNA sequencing using standard techniques.

### 1c Structured Polypeptides

Polypeptides tethered to a synthetic molecular structure are known in the art (Kemp, D. S. and McNamara, P. E., J. Org. Chem, 1985; Timmerman, P. et al., ChemBioChem, 2005). Meloen and co-workers had used tris(bromomethyl) benzene and related molecules for rapid and quantitative cyclisation of multiple peptide loops onto synthetic scaffolds for structural mimicry of protein surfaces (Timmerman, P. et al., ChemBioChem, 2005). Methods for the generation of candidate drug compounds wherein said compounds are generated by linking cysteine containing polypeptides to a molecular scaffold as for example tris(bromomethyl) benzene are disclosed in WO 2004/077062 and WO 2006/078161.

WO2004/077062 discloses a method of selecting a candidate drug compound. In particular, this document discloses various scaffold molecules comprising first and second reactive groups, and contacting said scaffold with a further molecule to form at least two linkages between the scaffold and the further molecule in a coupling reaction.

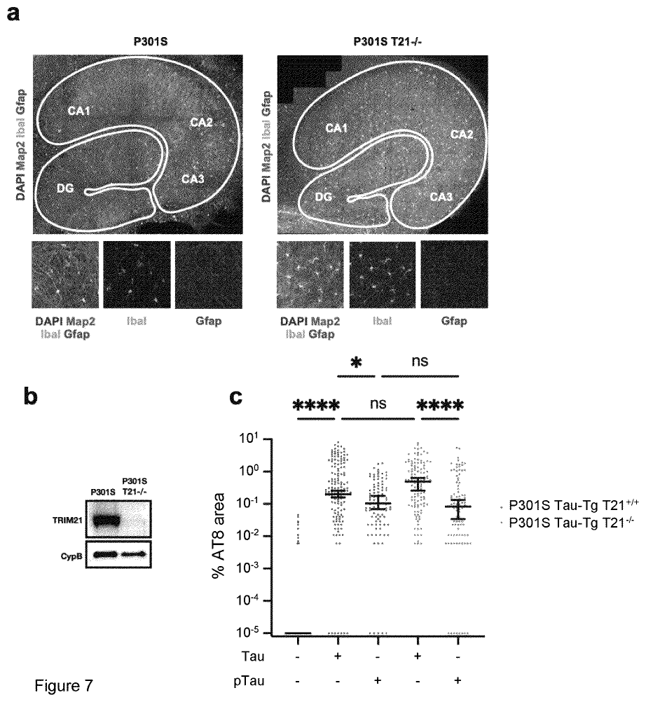

WO2006/078161 discloses binding compounds, immunogenic compounds and peptidomimetics. This document discloses the artificial synthesis of various collections of peptides taken from existing proteins. These peptides are then combined with a constant synthetic peptide having some amino acid changes introduced in order to produce combinatorial libraries. By introducing this diversity via the chemical linkage to separate peptides featuring various amino acid changes, an increased opportunity to find the desired binding activity is provided. FIG. 7 of this document shows a schematic representation of the synthesis of various loop peptide constructs.

International patent application WO2009098450 describes the use of biological selection technology, such as phage display, to select peptides tethered to synthetic molecular structures. In this approach, peptides are expressed on phage, and then reacted under suitable conditions with molecular scaffolds, such that a structurally constrained peptide is displayed on the surface of the phage.

Such structured peptides can be designed to bind to any desired antigen, and can be coupled to a RING domain in order to direct the antigen-ligand complex to the proteasome inside a cell.

1d. Small Molecules

Small molecule ligands are well known in the context of protac degradation molecules. For example, Silva et al (2019) proposed the use of a small molecule capable binding tau, previously used for PET imaging of tau, to promote tau degradation. Such small molecules, such F-AV-1451, may be useful in the context of the present invention. Various other tau PET tracers (Okamura 2019) and other small molecules that bind to specific forms of tau are available.

### 2. Tau

Tau is a microtubule-associated protein (MAP) present in normal mature neurons. It promotes of assembly and stability of microtubules. The biological activity of tau, primarily a neuronal protein, in promoting assembly and stability of microtubules is regulated by its degree of phosphorylation. Hyperphosphorylation of tau depresses its microtubule assembly activity and its binding to microtubules.

Human brain tau is a family of six proteins derived from a single gene by alternative mRNA splicing. These proteins differ in whether they contain three (T3L, T3S or T3) or four (14L, T4S or 14) tubulin binding domains (repeats, R) of 31 or 32 amino acids each near the C-terminal and two (T3L, T4L), one (T3S, T4S), or no (T3, T4) inserts of 29 amino acids each in the N-terminal portion of the molecule; the two amino-terminal inserts, 1 and 2, are coded by exon 2 and exon 3, respectively.

In Alzheimer disease (AD) and related disorders called tauopathies, tau is abnormally hyperphosphorylated and is accumulated as intraneuronal tangles of paired helical filaments (PHF), twisted ribbons and or straight filaments. The presence of these tangles directly correlates with dementia.

Antibodies or other ligands may be used to target Tau by selecting ligands for Tau binding according to any of the methods described herein. Antibodies specific for Tau are known in the art and are widely available commercially. Such antibodies can be modified to render them less able to bind Fc γ R, in accordance with the present invention.

Tau assemblies, seeds and aggregates are tau proteins of any of the six isoforms which are hyperphosphorylated, acetylated, truncated or otherwise modified to behave abnormally in the cell and are associated with neurodegenerative conditions. The terms seeds, assemblies and aggregates are used to denote the same thing and are interchangeable for the purposes of the invention.

### 3. Anti-Tau Antibodies

A number of anti-tau antibodies are known in the art and available form major suppliers of biological reagents. Several are also in clinical trials, mostly aimed at preventing uptake of tau to neurons though a blocking activity, promoting clearance to the periphery or promoting uptake to microglia via interactions with Fc γ Rs.

### 3a. Examples of Anti-Tau Antibodies

A list of antibodies suitable for tau therapy has been published in Ji and Sigurdsson, Drugs (2021) 81:1135-1152. The listed antibodies are currently in development for therapy of neurodegenerative conditions, and are suitable for use in conjunction with the present invention. See Table 1 of Ji and Sigurdsson, incorporated herein by reference.

### 4. Antibody Modification

Antibodies can be modified by introducing mutations into the sequence of the variable and constant domains. As noted above, it is established that mutations in the CDRs may be used to alter antibody specificity. Mutations in the Fc domain can, similarly, be used to alter effector functions and other properties of the antibody.

4a. Fc γ R binding

Biding to Fc γ R can be reduced by introducing mutations into the antibody Fc domain. Fc fragments may be modified to eliminate or substantially reduce the binding affinity for Fc γ receptors and complement (C1q). This modification prevents inflammatory responses. Formation of the Fc/F γ R complex recruits these effector cells to sites of bound antigen, typically resulting in signaling events within the cells and important subsequent immune responses such as release of inflammation mediators, B cell activation, endocytosis, phagocytosis, and cytotoxic attack. Avoiding these responses can be advantageous in neuronal immunotherapy. For example the Fc regions can be mutated (G236R/L328R) so that they do not bind Fc γ receptors. Other examples of mutations that substantially reduce or ablate binding to Fc γ receptors and complement include N297A or N297Q, D265A, L234A/L235A, L234A/L235A/P329G and N297A/L234A/L235A, among others identifiable by persons skilled in the art. A reduction in binding affinity for Fc γ receptors of at least 10-fold is preferred.

An overlapping but separate site on Fc serves as the interface for the complement protein C1q. In the same way that Fc/Fc γ R binding mediates ADCC, Fc/C1q binding mediates complement dependent cytotoxicity (CDC).

In addition to or supplementing amino acid modification, glycoform engineering may be used to modify the affinity and binding of Fc to Fc γ R. IgG has a single N-linked biantennary carbohydrate at Asn297 of the C_{H}2 domain. For IgG from either serum or produced ex vivo in hybridomas or engineered cells, the IgG are heterogeneous with respect to the Asn297 linked carbohydrate (Jefferis et al., 1998, Immunol. Rev. 163:59-76; and Wright et al., 1997, Trends Biotech 15:26-32). For human IgG, the core oligosaccharide normally consists of GlcNAc2Man3GlcNAc, with differing numbers of outer residues. The presence of fucose at this position reduces the affinity of Fc for the Fc receptor.

In certain embodiments, therefore, the antibodies of the present invention are modified to control the level of fucosylated oligosaccharides that are covalently attached to the Fc region. A variety of methods are well known in the art for generating modified glycoforms (Umaña et al., 1999, Nat Biotechnol 17:176-180; Davies et al., 2001, Biotechnol Bioeng 74:288-294; Shields et al., 2002, J Biol Chem 277:26733-26740; Shinkawa et al., 2003, J Biol Chem 278:3466-3473); (U.S. Pat. No. 6,602,684; U.S. Ser. No. 10/277,370; U.S. Ser. No. 10/113,929; PCT WO 00/61739A1; PCT WO G1/29246A1; PCT WO 02/31140A11 PCT WO 02/30954A1); (PotelligI™) technology [Biowa, Inc., Princeton, N.J.]; GIMAb™ glycosylation engineering technology [GLYCART biotechnology AG, Zurich, Switzerland]).

These techniques may be used to control the level of fucosylated oligosaccharides that are covalently attached to the Fc region, for example by expressing an antibodies in various organisms or cell lines, engineered or otherwise (for example Lec-13 CHO cells or rat hybridoma YB2/0 cells), by regulating enzymes involved in the glycosylation pathway (for example FUT8 [alpha] 1,6-fucosyltranserase]), or by modifying carbohydrate(s) after the antibody has been expressed.

Alternatively, antibodies may be rendered aglycosylated by mutating Asn297, thereby broadly affecting Fc γ R binding.

By “reduced affinity” as compared to a parent antibody as used herein is meant that a modified antibody binds an Fc γ R Fc receptor with significantly lower KA or higher K_{D} than the parent antibody when the amounts of variant and parent antibody in the binding assay are essentially the same. For example, the antibody variant with decreased Fc receptor binding affinity may display from about 5 fold to about 1000 fold, e.g. from about 10 fold to about 500 fold reduction in affinity in Fc receptor binding affinity compared to the parent antibody.

4b. TRIM Binding

Binding to TRIM21 can be increased using mutations in the Fc region of an antibody which increase the affinity for TRIM21. These are generally described in WO2017158421, incorporated herein by reference. Further mutations that increase affinity for TRIM21 are described in Ng et al and are incorporated herein by reference (Ng et al., 2019). WO2020106220 and WO2019235426 describe further mutants that increase affinity to TRIM21.

Exemplary mutations include mutations at positions 131, 256, 311, 345, 385, 433, 434, 435, 436 and/or 428, and 440. Particularly preferred is the mutation IgG1-Q311R/N434W/M428E or IgG1-T256P/H433T/N434R/Y435F/S440| with numbering according to the EU antibody sequence standard. Increasing the binding to TRIM21 increases the effectiveness of the therapeutic effect, as we have shown that this is mediated almost exclusively through TRIM21.

In some embodiments, improved TRIM21 activity can be obtained through means other than modifications which improve binding to TRIM21, for example due to changes in the hinge region (for example by extending the hinge region, and/or removal of hinge disulphide bridges) or an antibody in which the binding or activity of TRIM21 is increased by using a different antibody subtype or isotype (eg IgG3) or by incorporating the hinge region of one antibody subtype to another (eg IgG3 hinge into IgG1). Exemplary mutations for removal of disulphide bridges includes mutation of the three most N-terminal cysteines of the IgG3 hinge region to serine (IgG3Hinge-3S). This mutated hinge may be incorporated into other IgG classes including IgG1 and sometimes alongside the mutation S131C to ensure correct binding of the light chain (referred to as IgG1-S131C-IgG3Hinge-3S). IgG3 hinge is comprised of four exons, which may be deleted individually, or in combinations. These are referred to by the name of the exon that is deleted (eg IgG3_AHinge_exon_1)

Peptide sequence of IgG3 hinge with three cysteines in bold that are mutated to serine in IgG3Hinge-3S:

ELKTPLGDTTHTCPRCPEPKSCDTPPPCPRCPEPKSCDTPPPCPRCPEP
KSCDTPPPCPRCP

By “increased affinity”, as used herein, is meant that an antibody binds to Trim21 with a significantly higher equilibrium constant of association (KA) or lower equilibrium constant of dissociation (K_{D}) than the unmodified antibody when the amounts of variant and unmodified antibody in the binding assay are essentially the same. For example, an antibody with improved Trim21 binding affinity may display from about 5 fold to about 1000 fold, e.g. from about 10 fold to about 500 fold improvement in Trim 21 binding affinity compared to the unmodified antibody.

Exemplary mutations include M428L/N434S, M252Y/S254T/T256E, H433K/N434F, IgG1-Q311R/N434W/M428E, IgG1-Q311R/N434W, IgG1-Q311R, IgG1-N434W, IgG1-N434Y, IgG1-Q311H, IgG1-H433R, IgG1-E345R, IgG1-MST/G385E/M428E, IgG1-Y436W, IgG3 (b)-Q311R/N434F/H433R/R435H, IgG1-M428E, IgG1-Q311R/M428E, IgG1-Q311H/M428E, IgG1-G385E/M428E, IgG1-M428F, IgG1-N434K, IgG1-Q311F, IgG1-N434H, IgG3 (b)-Q311R/G385E/M428E/R435H, IgG1-Q311R/G385E/M428E, IgG-Q311R/N434Y/M428F, IgG1-T256P, IgG1-H433T, IgG1-N434R, IgG1-Y435F, IgG1-S4401, T256P/H433T/N434R/Y435F/S4401, IgG1-Q311R/G385E/M428E/N434Y, IgG1-Q311R/G385E/M428F/N434Y, IgG3-R435H, IgG1-N434F, IgG3-N434F/R435H, IgG1-S131C-IgG3Hinge, IgG1-S131C-IgG3Hinge-3S, IgG3-3S, IgG3-C131S-IgGIHinge, IgG1-IgG3Fc, IgG3-IgGIFc, IgG3_AHinge_exon_1, IgG3_AHinge_exon_1_2, IgG3_ΔHinge_exon 1_2_3, IgG3_ΔHinge_exon 1_2_3_4, IgG3_ΔHinge_exon 2, IgG3_ΔHinge_exon 2_3, IgG3_ΔHinge_exon 2_3_4, and IgG3-C131S/R435H-IgG1Hinge.

In one embodiment, affinity for Trim21 is increased by inserting into an IgG antibody a T256P mutation and FcγR binding is reduced by inserting into the same antibody a N297A mutation. T265P increases Fc affinity for Trim21 approximately 10-fold, and N297A prevents N-linked glycosylation at position 297, reducing FcγR binding and potentiating the Trim21 binding effect. FcγR binding can be further reduced by mutating the antibody as indicated in the preceding section. Advantageously, the antibody comprises T256P and N297A/L234A/L235A mutations. The antibody may comprise T256P and P329G/L234A/L235A mutations. Preferably, the antibody is an anti-Tau antibody.

In a further embodiment, the ability of an antibody to interact with Trim21 is increased by modifying or replacing the antibody hinge. In one embodiment, the ability of an IgG1 antibody to interact with Trim21 is increased by replacing the hinge region of the IgG1 isotype antibody with an IgG3 hinge. Preferably, the antibody is an anti-Tau antibody.

Hinge replacements and mutations may be combined, for instance in an IgG1 antibody including T256P as well as a hinge replacement with an IgG3 hinge.

4c. FcRn Binding

A site on Fc between the C_{H}2 and C_{H}3 domains mediates interaction with the neonatal receptor FcRn, as well as residues near the carboxy terminus of C_{H}3, the binding of which recycles endocytosed antibody from the endolysosome back to the bloodstream (Raghavan et al, 1996, Annu Rev Cell Dev Biol 12:181-220; Ghetie et al, 2000, Annu Rev Immunol 18:739-766).

The antibodies according to the invention preferably have FcRn binding maintained or enhanced. Modifications which increase FcRn binding include H433K/N434F; M252Y/S254T/T256E/H433K/N434F (see Vaccaro et al., Proc Natl Acad Sci USA. 2006 Dec. 5; 103 (49): 18709-18714).

Further potential mutations include M428L/N434S or T256D/T307Q (DQ) or T256D/T307W (DW), M252Y/T256D (YD), T307Q/Q311V/A383V, T256D/H286D/T307R/Q311V/A378A, and L309D/Q311H/N434S (DHS).

4d. Intracellular Stability

Modifications are known in the art which increase intracellular stability of antibodies or antibody fragments. In the case of antibodies and antibody fragments, CDR grafting and humanisation techniques can be used to combine antibody frameworks with known intracellular stability advantages with CDRs from desired target binding domains, in this case tau-binding antibodies (see for example EP 1 506 236).

In some cases, the stability of the hinge region in an intracellular environment can be increased by replacing the disulphide bonds formed by the cysteine residues in the hinge with other amino acids, including pairs and mixed pairs of Ala and Val amino acids (see for example Hagihara et al., BBA Volume 1844, Issue 11, November 2014, pp 2016-2023). Single chain antibodies, in which the domains are linked by a peptide linker, are also considered to show enhanced intracellular stability.

4e. Combinations

Antibodies may be further modified such that they incorporate more than one of the above properties. Examples include:
P329G, L234A, L235A, T256P, H433T, N434R, Y435F, S4401
P329G, L234A, L235A, T256P, H433K, N434R, Y435F, S4401
P329G, L234A, L235A, T256P, H433T, N434F, Y435F, S4401
P329G, L234A, L235A, T256P, H433K, N434F, Y435F, S4401
N297A, T256P, H433T, N434R, Y435F, S4401
N297A, T256P, H433K, N434R, Y435F, S4401
N297A, T256P, H433T, N434F, Y435F, S4401
N297A, T256P, H433K, N434F, Y435F, S4401
T256P, H433T, N434R, Y435F, S4401
IgG1-S131C-IgG3Hinge-3S, N297A, H433K, N434F
IgG1-S131C-IgG3Hinge-3S, N297A,
IgG1-S131C-IgG3Hinge-3S, P329G, L234A IgG1-S131C-IgG3Hinge-3S, P329G, L234A, H433K, N434F
P329G, L234A, M252Y, S254T, T256E
N297A, M252Y, S254T, T256E
IgG1-S131C-IgG3Hinge-3S P329G, L234A, L235A,
IgG1-S131C-IgG3Hinge-3S, N297A
IgG1-S131C-IgG3Hinge-3S, P329G, L234A, L235A, M252Y, S254T, T256E
IgG1-S131C-IgG3Hinge-3S, N297A, M252Y, S254T, T256E
T256P, N297A
T256P, P329G, L234A, L235A
T256P, N297A, L234A, L235A
T256P, N297A, L234A, L235A, M252Y, S254T, T256E
IgG1-IgG3Hinge, T256P
IgG1-IgG3Hinge, P329G, L234A, L235A
IgG1-IgG3Hinge, N297A, L234A, L235A
IgG1-IgG3Hinge, N297A
IgG1-IgG3Hinge, T256P, N297A, L234A, L235A.

### 5. Administration of Compounds

Generally, the compounds according to the invention will be utilised in purified form together with pharmacologically appropriate carriers. Typically, these carriers include aqueous or alcoholic/aqueous solutions, emulsions or suspensions, any including saline and/or buffered media. Parenteral vehicles include sodium chloride solution, Ringer's dextrose, dextrose and sodium chloride and lactated Ringer's. Suitable physiologically-acceptable adjuvants, if necessary to keep a polypeptide complex in suspension, may be chosen from thickeners such as carboxymethylcellulose, polyvinylpyrrolidone, gelatin and alginates.

Intravenous vehicles include fluid and nutrient replenishers and electrolyte replenishers, such as those based on Ringer's dextrose. Preservatives and other additives, such as antimicrobials, antioxidants, chelating agents and inert gases, may also be present (Mack (1982) Remington's Pharmaceutical Sciences, 16th Edition).

The compounds of the present invention may be used as separately administered compositions or in conjunction with other agents. These can include further antibodies, antibody fragments and conjugates, and various immunotherapeutic drugs, such as cylcosporine, methotrexate, adriamycin or cisplatinum, and immunotoxins. Pharmaceutical compositions can include “cocktails” of various cytotoxic or other agents in conjunction with the selected antibodies, receptors or binding proteins thereof of the present invention, or even combinations of selected polypeptides according to the present invention having different specificities, such as polypeptides selected using different target ligands, whether or not they are pooled prior to administration.

The route of administration of pharmaceutical compositions according to the invention may be any of those commonly known to those of ordinary skill in the art. For therapy, including without limitation immunotherapy, the selected antibodies, receptors or binding proteins thereof of the invention can be administered to any patient in accordance with standard techniques. The administration can be by any appropriate mode, including parenterally, intravenously, intramuscularly, intraperitoneally, transdermally, via the pulmonary route, or also, appropriately, by direct infusion with a catheter. The dosage and frequency of administration will depend on the age, sex and condition of the patient, concurrent administration of other drugs, counterindications and other parameters to be taken into account by the clinician.

The compounds of this invention can be lyophilised for storage and reconstituted in a suitable carrier prior to use. This technique has been shown to be effective and art-known lyophilisation and reconstitution techniques can be employed. It will be appreciated by those skilled in the art that lyophilisation and reconstitution can lead to varying degrees of activity loss and that use levels may have to be adjusted upward to compensate. The compositions containing the present peptide ligands or a cocktail thereof can be administered for prophylactic and/or therapeutic treatments. In certain therapeutic applications, an adequate amount to accomplish at least partial inhibition, suppression, modulation, killing, or some other measurable parameter, of a population of selected cells is defined as a “therapeutically-effective dose”. Amounts needed to achieve this dosage will depend upon the severity of the disease and the general state of the patient's own immune system, but generally range from 0.005 to 5.0 mg of selected peptide ligand per kilogram of body weight, with doses of 0.05 to 2.0 mg/kg/dose being more commonly used. For prophylactic applications, compositions containing the present peptide ligands or cocktails thereof may also be administered in similar or slightly lower dosages.

A composition containing a compound according to the present invention may be utilised in prophylactic and therapeutic settings to aid in the alteration, inactivation, killing or removal of a select target cell population in a mammal. In addition, the selected repertoires of polypeptides described herein may be used extracorporeally or in vitro selectively to kill, deplete or otherwise effectively remove a target cell population from a heterogeneous collection of cells. Blood from a mammal may be combined extracorporeally with the selected peptide ligands whereby the undesired cells are killed or otherwise removed from the blood for return to the mammal in accordance with standard techniques.

Orally administered compositions according to the invention, for example ProTacs, optionally consist essentially of the functional ingredients and suitable pharmaceutically acceptable carriers and/or excipients.

The phrases “physiologically acceptable carrier” and “pharmaceutically acceptable carrier”, which may be interchangeably used, refer to a carrier or a diluent that does not cause significant irritation to an organism and does not abrogate the activity (e.g., biological activity) and properties of the functional ingredient (e.g., a therapeutically active agent).

The term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

The term “unit dosage form” describes physically discrete units, each unit containing a predetermined quantity of one or more active ingredient(s) calculated to produce the desired therapeutic effect, in association with at least one pharmaceutically acceptable carrier, diluent, excipient, or combination thereof.

In some embodiments of any one of the embodiments described herein, the composition is formulated as a solid composition. In some embodiments, the composition is formulated as a tablet.

Techniques for formulation and administration of drugs may be found in “Remington's Pharmaceutical Sciences,” Mack Publishing Co., Easton, Pa., latest edition, which is incorporated herein by reference.

A therapeutically effective amount of a compound as described herein used in the present invention may vary depending upon the route of administration and dosage form. Effective amounts of invention compounds typically fall in the range of about 0.001 up to 100 mg/kg/day, and more typically in the range of about 0.05 up to 10 mg/kg/day. Typically, the compound or compounds used in the instant invention are selected to provide a formulation that exhibits a high therapeutic index. The therapeutic index is the dose ratio between toxic and therapeutic effects which can be expressed as the ratio between LD50 and ED50. The LD50 is the dose lethal to 50% of the population and the ED50 is the dose therapeutically effective in 50% of the population. The LD50 and ED50 are determined by standard pharmaceutical procedures in animal cell cultures or experimental animals.

The instant invention also provides for pharmaceutical compositions and medicaments which may be prepared by combining one or more compounds described herein, pharmaceutically acceptable salts thereof, stereoisomers thereof, tautomers thereof, or solvates thereof, with pharmaceutically acceptable carriers, excipients, binders, diluents or the like to inhibit or treat primary and/or metastatic prostate cancers. Such compositions can be in the form of, for example, granules, powders, tablets, capsules, syrup, suppositories, injections, emulsions, elixirs, suspensions or solutions. The instant compositions can be formulated for various routes of administration, for example, by oral, parenteral, topical, rectal, nasal, or via implanted reservoir. Parenteral or systemic administration includes, but is not limited to, subcutaneous, intravenous, intraperitoneal, and intramuscular injections. The following dosage forms are given by way of example and should not be construed as limiting the instant invention.

For oral, buccal, and sublingual administration, powders, suspensions, granules, tablets, pills, capsules, gelcaps, and caplets are acceptable as solid dosage forms. These can be prepared, for example, by mixing one or more compounds of the instant invention, or pharmaceutically acceptable salts or tautomers thereof, with at least one additive such as a starch or other additive. Suitable additives are sucrose, lactose, cellulose sugar, mannitol, maltitol, dextran, starch, agar, alginates, chitins, chitosans, pectins, tragacanth gum, gum arabic, gelatins, collagens, casein, albumin, synthetic or semi-synthetic polymers or glycerides. Optionally, oral dosage forms can contain other ingredients to aid in administration, such as an inactive diluent, or lubricants such as magnesium stearate, or preservatives such as paraben or sorbic acid, or antioxidants such as ascorbic acid, tocopherol or cysteine, a disintegrating agent, binders, thickeners, buffers, sweeteners, flavoring agents or perfuming agents. Tablets and pills may be further treated with suitable coating materials known in the art.

Liquid dosage forms for oral administration may be in the form of pharmaceutically acceptable emulsions, syrups, elixirs, suspensions, and solutions, which may contain an inactive diluent, such as water. Pharmaceutical formulations and medicaments may be prepared as liquid suspensions or solutions using a sterile liquid, such as, but not limited to, an oil, water, an alcohol, and combinations of these. Pharmaceutically suitable surfactants, suspending agents, emulsifying agents, may be added for oral or parenteral administration. As noted above, suspensions may include oils. Such oils include, but are not limited to, peanut oil, sesame oil, cottonseed oil, corn oil and olive oil. Suspension preparations may also contain esters of fatty acids such as ethyl oleate, isopropyl myristate, fatty acid glycerides and acetylated fatty acid glycerides. Suspension formulations may include alcohols, such as, but not limited to, ethanol, isopropyl alcohol, hexadecyl alcohol, glycerol and propylene glycol. Ethers, such as but not limited to, poly(ethyleneglycol), petroleum hydrocarbons such as mineral oil and petrolatum; and water may also be used in suspension formulations.

### Examples

### Methods

### Cell Culture

HEK293 cells were maintained in complete DMEM with 10% vol/vol fetal calf serum (FCS), 100 U/ml penicillin and 100 μg/ml streptomycin at 37° C. in a 10% CO₂ humid atmosphere. Hybridoma cells were cultured in OptiMEM supplemented with 2% FCS in cell factory systems (Thermo Fisher Scientific) and the supernatant was harvested twice-weekly. Supernatant was filtered through a 0.22 μm 500 ml filter units (Thermo Fisher Scientific) and stored at 4° C. before purification.

### Mice and In Vivo Immunotherapy

All animal work was licensed under the UK Animals (Scientific Procedures) Act 1986 and was approved by the Medical Research Council Animal Welfare and Ethical Review Body.

C57BL/6 Trim21^{-/-} mice (MGI: 3849316) were obtained from Jackson Laboratories. P301S tau-transgenic mice (Allen et al., 2002a) (MGI: 3778191), which express ON4R tau under the control of a Thy1 promoter, were extensively backcrossed to C57CL/6. The strains were bred by backcrossing for eight generations. Through the course of the study, animals were weighed and observed twice daily for clinical signs including subdued behaviour, pilo-erection, hunched posture, ataxia and paresis. Animals that displayed clinical signs that did not improve within a 6-hour period were sacrificed. 20 days old P301S tau transgenic mice and age-matched Trim21^{-/-}-P301S tau transgenic mice were injected weekly (intraperitoneal or i.p.) for 60 days with either 30 mg/kg of mAb AP422, 30 mg/kg of anti-AdV hexon antibody 9C12, or treated with PBS. Mice were observed for the duration of the protocol as above. Post exsanguination, the lumbar regions of the spinal cords were harvested and snap frozen in liquid nitrogen for downstream biochemical and tau seeding analyses. 4 week old homozygous human P301S tau transgenic mice on a pure C57BL/6 JAX and age-matched Trim21^{-/-} ON4R P301S tau transgenic on the same background mice were injected weekly (intraperitoneal or i.p.) for 17 weeks with either 30 mg/kg of AP422, 30 mg/kg of a commercially available mouse polyclonal IgG reactive to total tau (mIgG-T; ImmunoReagents, Inc.), or left untreated. Post exsanguination, whole brains including the brainstem and cerebellum were snap frozen for downstream biochemical analyses.

### Organotypic Hippocampal Slice Culture

Organotypic hippocampal slice cultures were prepared and cultured as described previously (Miller et al., 2021). Brains from P6-P9 pups were rapidly removed and kept in ice-cold Slicing Medium (EBSS+25 mM HEPES) on ice. All equipment was kept ice-cold. Brains were bisected along the midline and the cerebellum was removed using a sterile scalpel. The medial cut surface of the brain was adhered to the stage of a Leica VT1200S Vibratome using cyanoacrylate (Loctite Super Glue) and the vibratome stage was submerged in ice-cold Slicing Medium. Hemispheres were arranged such that the vibratome blade sliced in a rostral to caudal direction. Sagittal slices of 300 μm thickness were prepared and the hippocampus was sub-dissected using sterile needles. Hippocampal slices were transferred to 15 ml tubes filled with ice-cold Slicing Medium using sterile plastic pipettes with the ends cut off. Slices were then transferred onto sterile 0.4 μm pore membranes (Millipore PICMORG50) in 6-well plates pre-filled with 1 ml pre-warmed Culture Medium (50% MEM with GlutaMAX, 18% EBSS, 6% EBSS+D-Glucose, 1% Penicillin-Streptomycin, 0.06% nystatin and 25% Horse Serum) and incubated at 37° C. in a humid atmosphere with 5% CO₂. Three slices were typically maintained per well. 24 h after plating 100% media was exchanged and thereafter a 50% media exchange was carried out twice per week. For seeding experiments, tau assemblies were diluted in 1 ml Culture Medium and added to the underside of the membrane with 100% media change. Where antibodies were used, tau assemblies were mixed with antibodies or buffer only at 1:5 for 1 h before dilution to 50 nM in Culture Medium and application to OHSCs. After three days, assemblies were removed by 100% media change. Alternatively, 20 μl of tau assemblies diluted in Culture Medium was applied directly to OHSCs on the apical side.

Extraction of Tau Assemblies from Mouse Brains, Spinal Cords and OHSCs

Insoluble tau was extracted from brain, spinal cords and OHSCs using the sarkosyl extraction protocol (Goedert et al., 1992) as with modifications as previously (Miller et al., 2021). Briefly, tissues were homogenised in ice-cold H-Buffer (10 mM Tris pH 7.4, 1 mM EGTA, 0.8 M NaCl, 10% sucrose, protease and phosphatase inhibitors (Halt™ Protease and Phosphatase Inhibitor Cocktail)) using the VelociRuptor V2 Microtube Homogeniser (Scientific Laboratory Supplies). The homogenates were spun for 20 min at 20,000×g and supernatant collected. Sarskosyl was added to a final concentration of 1% to the supernatants and incubated for 1 h at 37° C. Supernatants were then centrifuged at 100,000×g at 4° C. for 1 h. The resulting pellet was resuspended in 0.2 volumes (weight of tissue) of TBS and sonicated for 15 s in a water-bath sonicator before storage at −80° C. for immunoblotting and tau seeding assays.

### Seeding Assay in HEK293

Seeding assays were carried out largely as described previously (McEwan et al., 2017b). HEK293 cells expressing P301S tau-venus were plated at 15,000 cells per well in black 96-well plates in 50 μL OptiMEM (Thermo Fisher). Tau assemblies were diluted in 50 μL OptiMEM (Thermo Fisher) and added to cells with 0.5 μl per well Lipofectamine 2000 (Thermo Fisher). After 1 h, 100 μL complete DMEM was added to each well to stop the transfection process. Cells were incubated at 37° C. in an IncuCyte® S3 Live-Cell Analysis System for 48-72 h after addition of fibrils. Tau-venus aggregates were quantified using ComDet plugin in ImageJ.

### Production of Antibodies

cDNA encoding the constant domains of WT mIgG2a and the variable heavy chains of AP422 were synthesised and subcloned into pLNOH2 vectors (Norderhaug et al., 1997) with ampicillin resistance by GenScript Biotech Corporation. Corresponding murine kappa light chains were synthesised and subcloned into separate pLNOH2 vectors. Effector-silencing mutations corresponding to codons encoding P329G, L234A, L235A1 of the heavy chain constant domains were introduced by site-specific mutagenesis. Resulting expression vectors were co-transfected into Expi293F™ cells (Thermo Fisher Scientific, A14527) using an ExpiFectamine™ 293 Transfection Kit (Thermo Fisher Scientific, A14524) according to the manufacturer's instructions. Abs were collected as supernatant 6 days post-transfection and purified on a CaptureSelect™ mouse LC-kappa Affinity Matrix (Thermo Fisher Scientific, 191315005). Protein fractions were eluted with 0.1 M glycine-HCl (pH 2.7) and neutralised by adding 1M Tris-HCl (pH 8.0). Eluates were concentrated and buffer-exchanged into PBS on 50K Amicon® Ultra-15 Centrifugal Filter Units (Merck Millipore, UFC905096) followed by size-exclusion chromatography to isolate monomeric fractions using a Superdex™ 200 10/300 GL column (Cytiva) coupled to an Äkta Avant 25 (Cytiva). Eluted monomeric Abs were concentrated on 50K Amicon® Ultra-4 Centrifugal Filter Units (Merck Millipore, UFC810024) and subjected to SDS-PAGE using a Bolt™ 12% Bis-Tris polyacrylamide gel (Thermo Fisher Scientific, NW00125BOX) to evaluate protein integrity. Abs from hybridoma were purified from culture supernatant on Protein G HiTrap HP column (Cytiva) coupled to an Äkta Pure system (Cytiva). Protein was eluted using 0.1 M glycine (pH 2.7) and neutralised in 1M Tris-HCl (pH 9.0). Antibodies were buffer exchanged to PBS using 12000 MWCO SpectraPor membranes and concentrated on Vivaspin 50,000 MWCO Centrifugal Concentrators (Cole-Parmer). All Abs were snap frozen for storage at −80° C.

### Tau Production

The expression and purification of recombinant human ON4R tau bearing the P301S mutation from E. coli BL-21 (DE3, Agilent Technologies) was performed as described previously (Goedert and Jakes, 1990) with small modifications. Bacterial pellets were collected through centrifugation (3300 g, 4° C., 10 min) and then resuspended in 10 ml/L of culture with buffer A (50 mM MES pH 6.5, 10 mM EDTA, 14 mM B-mercaptoethanol, 0.1 mM PMSF, 1 mM benzamidine, 1× complete EDTA-free protease inhibitors). The resuspended bacteria were lysed on ice using a probe sonicator and boiled for 10 min at 95° C. which denatures the majority of proteins, but not tau. Denatured proteins were pelleted by ultracentrifugation at 100,000 g, 4° C. for 50 min. The clarified supernatant containing monomeric tau P301S was then passed through a HiTrap CaptoS (Cytiva) cation exchange column and the bound proteins were eluted through a 0-50% gradient elution with Buffer A containing 1 M NaCl. Eluted fractions were assessed through SDS-PAGE and total protein staining with Coomassie InstantBlue. Fractions of interest were concentrated using 10 kDa cut-off Amicon Ultra-4 concentrators (Merck Millipore) before loading on a Superdex 200 10/300 GL (Cytiva) size exclusion chromatography column. All purification was performed on an ÄKTA Pure system (Cytiva). The final tau P301S protein was stored in PBS containing 1 mM DTT.

### Tau Phosphorylation

Recombinant tau assemblies at 12 μM were treated with ERK2 (Abcam), which is a confirmed kinase of S422 (Yoshida et al., 2004). Reactions were performed in the presence of 100 mM ATP and Halt protease inhibitors in TBS at 30° C. overnight. Phosphorylation of the S422 site was confirmed by dot blot using AP422.

Preparation of Tau from Human Brain

Tau filaments were obtained from anonymized postmortem tissue donated by patients to the Cambridge Brain Bank under the ethically approved protocol for “Neurodegeneration Research in Dementia” (REC 16/WA/0240). The 4 donors were a 74 year-old female with clinical and pathologically confirmed diagnosis of corticobasal degeneration; a 85 year-old male with clinical and pathologically confirmed diagnosis of progressive supranuclear palsy; a 79 year-old male with a clinical diagnosis of dementia and pathologically confirmed Alzheimer's disease Braak Stage VI, and a 37 year-old male dying of renal failure secondary to type 1 diabetes and no neuropathology (control). 2 g of cortical grey matter was extracted according to a modified version of the method of Guo et al. Briefly, fresh-frozen cortical gray matter was homogenized in 9 volumes of extraction buffer (10 mm Tris-HCl [pH 7.5], 0.8 m NaCl, 10% sucrose, 1 mm EDTA, 0.1 mm PMSF, 0.1% Sarkosyl, 2 mm imidazole, 1 mm NaV, 1 mm NaF, 2 mm DTT, Complete Ultra EDTA-free protease inhibitor mixture [Roche]) using a VelociRuptor V2 homogenizer and tubes prefilled with 2.8-mm acid-washed stainless steel beads. Homogenate was spun at 10,000×g for 10 min at 4° C. and filtered through a 50-μm cell strainer. The pellet was reextracted with a further 4.5 volumes of extraction buffer and homogenized and clarified as above. Filtered supernatants were combined, and Sarkosyl was added to a final concentration of 1% before stirring at 100 rpm for 1 h. Samples were then subjected to ultracentrifugation at 100,000×g for 75 min at 4° C. The supernatant was separated from the pellet, and the latter was rinsed with PBS before resuspension and vortexing to break it apart. The resuspended pellet was further diluted in PBS and then centrifuged at 130,000×g for 1 h at 4° C. The resulting pellet was resuspended in 100 μl per gram gray matter and broken apart by 16 h of agitation at room temperature and passing through 18-, 23-, and 26-gauge needles. The resuspended pellet was sonicated (Hielsher S26D11X10 Vial-Tweeter Sonotrode at settings A 100%, C 50%, and 200 Ws). The sample was then centrifuged at 100,000×g for 40 min at 4° C. The pellet was resuspended again in 50 μl PBS per gram of gray matter and subjected to breaking apart using needles and sonication as above. Finally, the sample was subjected to a clearing spin at 10,000×g at 4° C. The concentrated Tau filaments were stored at −80° C. prior to use.

### Fc-receptor ELISA

To evaluate FcγR binding, 96-well plates were coated with titrated amounts (5000-40 ng/ml) of Ab variants diluted in PBS at a volume of 100 μL per well. Following overnight incubation at 4° C., plates were washed 4 times using PBS with 0.05% Tween20 (T) and blocked with 250 μL of PBS-T containing 4% skimmed milk (M) at room temperature (RT) for 1 h. Between all subsequent layers, plates were washed as previously described. Next, biotinylated recombinant soluble murine FcγRI (Sino Biological, 158-50086-M27H-B-100), FcγR2b (Sino Biological, 158-50030-M27H-B-100), FcγRIII (Sino Biological, 158-50326-M27H-B-100) and FcγRIV (Sino Biological, 158-50036-M27H-B-100) were incubated with streptavidin-AP conjugate (Roche, 11089161001) at a 1:1 molar ratio for 20 min at RT and added to the plate at final concentrations of 0.25 μg/mL FcγRs and 3.36 μg/ml streptavidin-AP. After 1 h of incubation on a shaker at RT, FcγR binding was visualised by adding 100 μL of 10 μg/mL Phosphatase substrate (Sigma-Aldrich, S0942) dissolved in diethanolamine solution (pH 9.8). Absorbance was measured at 405 nm with a Sunrise spectrophotometer (Tecan).

Human iPSC Experiments

Naïve human iPSCs gene edited to include doxycycline-inducible NGN2 transcription factor (iNeurons(Fernandopulle et al., 2018)) were maintained in E8 medium (Stem Cell Technologies) on vitronectin (Thermo Fisher) coated plates. iPSCs were passaged with 4 mM EDTA or Accutase (ThermoFisher) and ROCK inhibitor Y-27632 (BD Biosciences) when 70% confluency was reached. ROCK inhibitor at 10 UM was used for every passage of iPSC and iPSC-derived neurons, and removed the following day. Differentiation into cortical neurons was performed according to modified versions of existing experimental protocols (Fernandopulle et al., 2018). In brief, iPSCs were differentiated on Geltrex coated plates using DMEM/F-12 media supplemented with non-essential amino acids (NEAA) (1×), P/S (1×), glutamine (Q) (1×), N2 supplement (1×), 50 μM 2-Mercaptoethanol and Doxycycline (Dox) (2 μg/ml) for the first two days. For Differentiation from days 3-14, Neurobasal media was supplemented with penicillin-streptomycin (1×), L-Glutamine (1×), B-27 supplement (1×), NT-3 (10 ng/ml), 2-Mercaptoethanol (50 μM), Dox (2 μg/ml) and BDNF (10 ng/ml). Full media changes were performed daily until day 6, after which half-media changes were performed every other day. At day 3, the neurons were dissociated into single cells using Accutase and seeded onto Geltrex coated plates. Cells were seeded into 12-well plates at 1 million cells/well for western blotting, or into 96-well plates at 40,000 cells/well for Adenovirus neutralisation assays. DIV13 neurons were treated with human IFN-α (Sigma-Aldrich, SRP4596) at 5000 IU/mL for 16h before lysis in appropriate volume of 1× RIPA buffer (Sigma-Aldrich, R0278). For adenovirus infection experiments, adenovirus type 5 vector expressing eGFP under the human synapsin promoter, Ad-SYN-GFP (Signagen, SL100718) was mixed with humanised anti-hexon antibody rh9C12 IgG1, PBS or IgG1-H433A at 70 μg/mL, and incubated for 1 h to allow binding to reach equilibrium. Complexes were added to DIV14 neurons by dilution at 1:20 into media. After 48h incubation at 37° C., cells were dissociated with Accutase into a single cell suspension. GFP-positive cells were analysed by flow cytometry (CytoFlex).

### Western Blot

Lysates were cleared by centrifugation and resuspended with 4× NuPAGE LDS sample buffer (Thermo Fisher, NP0007) with 2 mM B-mercaptoethanol, before boiling for 5 minutes. Samples were subjected to SDS-PAGE using NuPAGE Bis-Tris 4-12% gels (Thermo Fisher, NP0324BOX) and transferred to 0.2 μm PVDF membrane using the Bio-Rad Transblot Turbo Transfer System. The membrane was blocked in 5% milk or 5% NGS with 0.2× fish gelatin in TBS-T (0.1% Tween-20 in TBS) for 1 h at room temperature before incubation with primary antibodies directed against human Trim21 (Santa Cruz Biotechnology, sc-25351), CypB (Santa Cruz Biotechnology, sc-130626), STAT1 (Cell Signalling Technology, 9172) and PSD-95 (Millipore, MABN68), phospho-tau ((Ser202, Thr205), AT8, Thermo Fisher, MN1020), pan-tau monoclonal antibody (HT7, Thermo Fisher Scientific, MN1000). Membranes were incubated in primary antibody overnight at 4° C. and following repeated washes with TBS-T, were incubated with secondary HRP/Alexa-Fluor conjugated antibodies for 1 h at room temperature. Membranes were washed with TBS-T and incubated with HRP substrate (Millipore, WBKLS0500) before imaging with the ChemiDoc system (BioRad).

### Dot Blot

Samples were transferred onto a 0.2-μm nitrocellulose membrane (Roti-NC transfer membrane, Carl Roth) using the Bio-Dot apparatus (Bio-Rad). Subsequently, the membrane was immunoblotted with the indicated antibody before probing with secondary antibodies conjugated to Alexa488/555/647 fluorophores and further processed as described above.

### Adenovirus Neutralisation Assay

HEK293T WT or TRIM21 KO cells were plated at 1×10⁵cells per well in 24-well plates and allowed to adhere overnight. AdV5-GFP was mixed with antibody at the indicated concentration for 1 hour at RT to allow complex formation. 20 μl of virus:antibody complexes were added per 500 μl of DMEM per well and incubated for 24 hours at 37° C. After infection, cells were collected by trypsinisation and GFP infection was analysed via flow cytometry using a Cytoflex machine. Fold neutralisation was calculated by: dividing % infection virus only by % infection with respective antibody concentration. 9C12 AdV Hexon ELISA Recombinant AdV5 hexon protein (Abcam) was diluted to 1 μg/ml in PBS saline and coated an 96-well plate (ELISA plates) O/N at 4° C. Remaining surface area was blocked with PBS+4% milk, before washing 4× times with PBS+0.05% Tween 20. Titrated amounts of 9C12 antibody diluted in PBS+0.05% Tween 20 and 4% milk (PBS/M/T) were incubated for 1 hour at RT. After washing as above, a HRP-conjugated anti-human kappa LC from goat (Abcam, diluted 1:3000 in PBS/M/T) was added and incubated for 1 hour at RT. Binding was visualized by addition of tetramethylbenzidine solution and the reaction was stopped by the addition of 0.16 nM sulfuric acid. 450-nm absorption values were recorded using a BMG Clariostar reader.

TRIM21 PRYSPRY production 6×His human TRIM21 PRYSPRY was expressed in E. coli (C41 strain) and purified using Nickel affinity chromatography and Size Exclusion Chromatography (SEC). Briefly, cells were grown in 2×TY (supplemented with 0.5% glucose, 2 mM MgSO4 and appropriate antibiotics) at 37° C. for 2-3 h (OD600 around 0.6-1), after which they were induced with 1 mM IPTG and incubated at 18° C. overnight. Cells were pelleted with a Sorvall SLC-6000 compatible centrifuge at 4500× g for 25 min and the pellet snap frozen until processed. The pellet was resuspended in lysis buffer (50 mM Tris pH 8, 1 M NaCl, 10% v/v BugBuster (Merck, Gillingham, UK), 10 mM imidazole, 2 mM DTT and 1× complete protease inhibitors (Roche, Basel, Switzerland) and sonicated for 15 min total time (10 s on/20 s off) at 70% amplitude. The soluble fraction was recovered by centrifugation at 40,000×g in a JLA25.50 rotor and put through a gravity flow column with 5 mL of NiNTA Agarose (Qiagen). The bound fraction was washed in Buffer B (300 mM NaCl, 50 mM Tris pH 8, 10 mM imidazole and 1 mM DTT) and eluted with Buffer E (300 mM NaCl, 50 mM Tris pH 8, 400 mM imidazole and 1 mM DTT). Fractions containing the protein were pooled, filtered, and separated by SEC using HiLoad 26/600 Superdex 75 pg column (Cytiva, Marlborough, MA, USA) in 150 mM NaCl, 50 mM Tris pH 8 and 1 mM DTT. The appropriate fractions were pooled and concentrated to 10-15 mg/mL.

### Human TRIM21 PRYSPRY Fluorescence Anisotropy

Recombinant 6×His human TRIM21 PRYSPRY was labelled using Alexa Fluor 488 Microscale Protein Labeling Kit (A30006), following the manufacturer's instructions. Following labelling, 5 nM labelled PRYSPRY was mixed with titrated antibodies in PBS+0.01% Tween 20 for 20 minutes at RT. Polarisation signal was read on a BMG Clariostar plate reader (excitation 485 nm, emission filter for channel A 520 nm, emission filter for channel B 520 nm).

### 3B2 Seeding and Neutralisation Assay

HEK293 cells expressing P301S tau-venus were plated at 20,000 cells per well in black 96-well plates in 50 μL OptiMEM (Thermo Fisher). Tau assemblies were mixed with antibodies or buffer only at 1:5 for 1 h before dilution to 0.25 nM tau assemblies in OptiMEM and added to cells with 0.5 μl per well Lipofectamine 2000 (Thermo Fisher). After 1 h, 100 μL complete DMEM was added to each well to stop the transfection process. Cells were incubated at 37° C. for 72 h after addition of fibrils. Tau-venus aggregates were quantified using the Nikon microscol

### HTRF FcγR1 and FcRn Binding Assay

The CD64 (FcγRI) Cellular Binding Assay (6FC64PAG) and FcRn Binding Assay (64FCRNPET) were performed according to the manufacturer's instructions and read on the BMG Clariostar.

### Human Microglia Inflammation Assay

Human iPSC differentiation to microglia and maintenance was performed as previously described (Washer et al., 2022). Microglia cultures were treated with sarkosyl insoluble tau extracted from HEK293 cell expressing aggregated human tau. Tau at 0.05 nM was incubated with AP422 antibody at 250 nM for 1 hr at RT. Complexes were diluted 10-fold in media, before addition to the cells for 24 hr. Total media was collected from each well and used undiluted in the human TNF-alpha ELISA Kit (R&D Systems, DY210-05), which was performed according to the manufacturer's instructions.

### Example 1

### Some Anti-Tau Antibodies Fail to Block Entry to the Cytosol

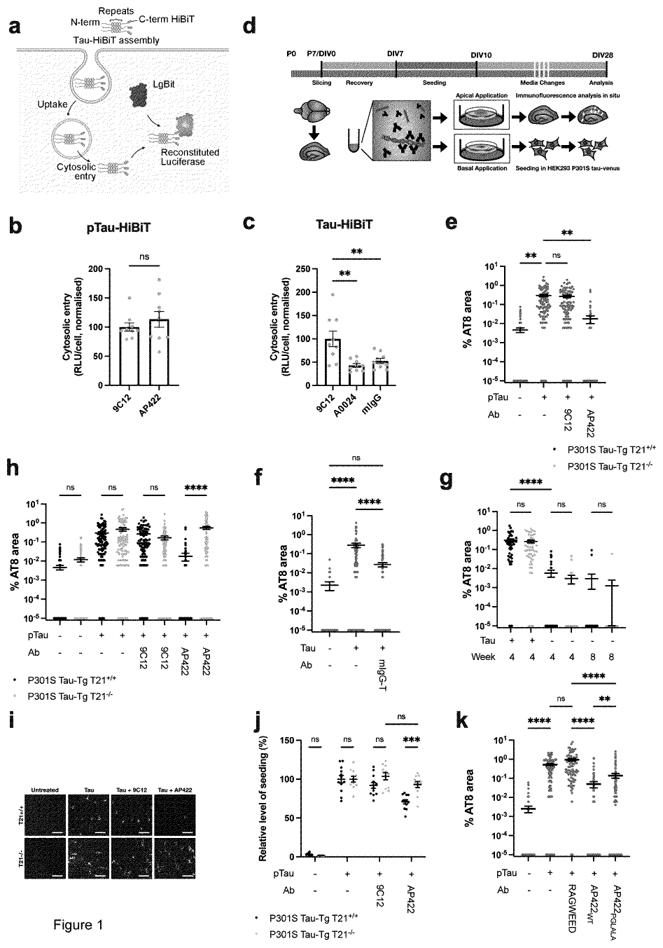

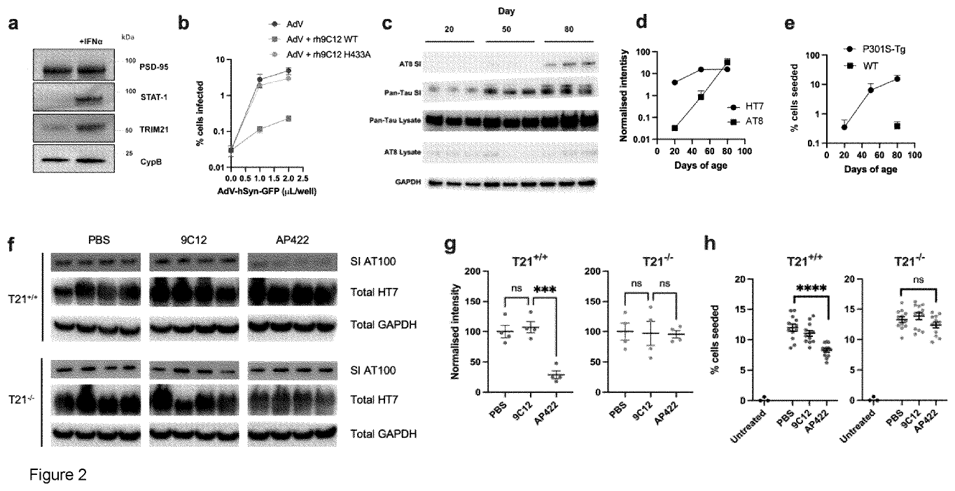

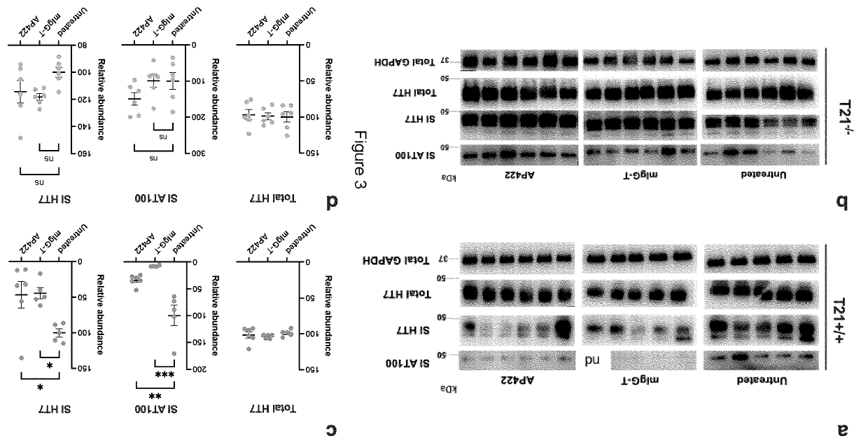

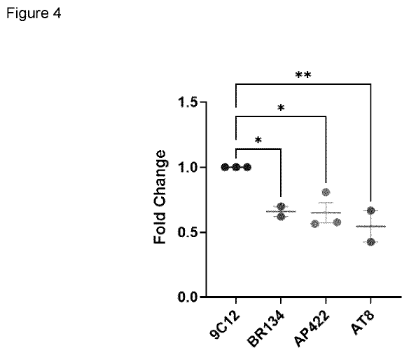

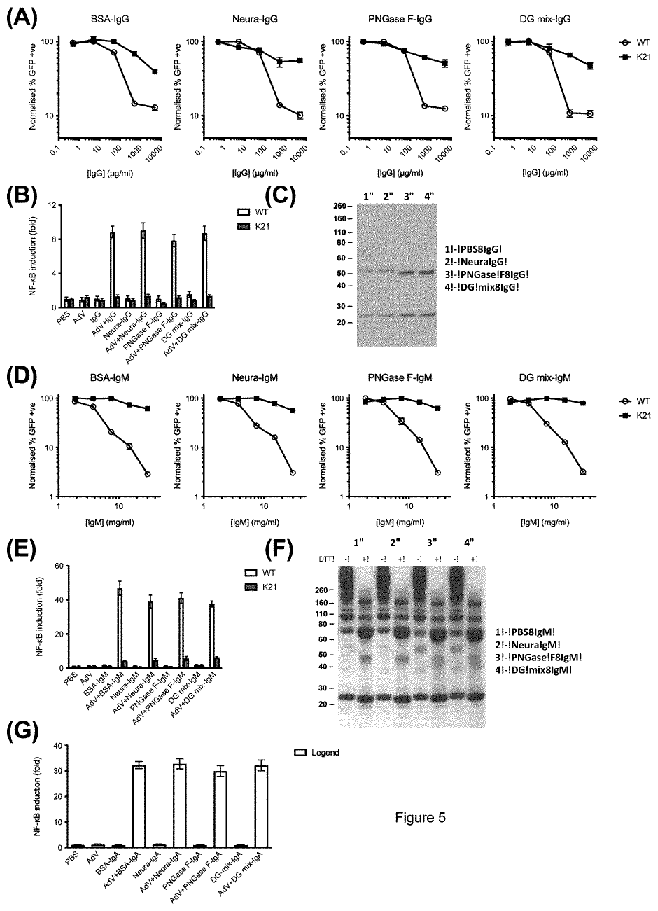

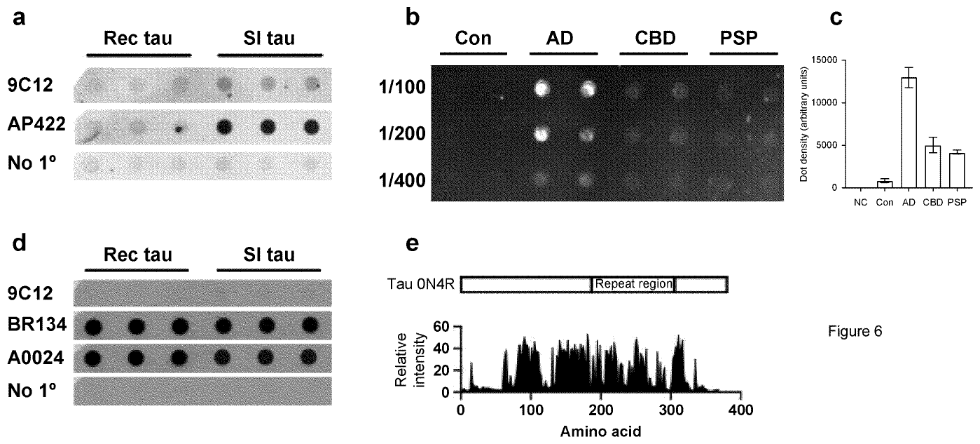

We first sought to determine the role of entry-blocking versus intracellular mechanisms in the neutralisation of seeded tau aggregation. To measure entry of tau to the neuronal cytosol, we used luciferase complementation between full-length ON4R tau expressed in fusion with an 11 amino acid tag (HiBiT) and a luciferase fragment (LgBiT) expressed in the cytosol of primary neurons (FIG. 1a). The assay reports on the LRP1 and HSPG-dependent entry of tau assemblies to the cytosol (Tuck et al., 2021). We incubated tau-HiBiT assemblies with control or tau-specific antibodies prior to addition to neurons. Mouse monoclonal IgG1 AP422 binds tau phosphorylated at S422 (Hasegawa et al., 1996) and recognises tau prepared from Alzheimer's disease, corticobasal degeneration and progressive supranuclear palsy (FIG. 6). AP422 failed to prevent entry of phospho-tau assemblies to the cytosol when compared to control Abs (FIG. 1b). Dako-A0024, which binds to the microtubule repeat region that interacts with surface receptors HSPGs and LRP1, reduced tau entry by ˜50% (FIG. 1c). A mouse polyclonal IgG preparation, mIgG-T, that epitope mapping revealed binds multiple epitopes of tau including the repeat domain, also reduced tau entry. These data show that antibodies block entry of tau to the cytosol in an epitope-dependent manner, and that some anti-tau Abs fail to inhibit entry.

We next examined whether antibodies could be internalised to neurons in complex with tau in order to contact TRIM21. We prepared mixed neural cultures from Trim21-1-mice and treated them with AAV1/2 expressing mCherry-labelled mouse TRIM21. We confirmed that this treatment led to the expression of the mCherry-TRIM21 construct by western blot (FIG. 13c). We treated these cells with recombinant tau assemblies either alone, or in complex with BR134, a polyclonal rabbit antibody raised against the tau C-terminus and previously shown to exert neutralisation via TRIM21 (McEwan et al., 2017). We confirmed that BR134 was able to interact with the mouse TRIM21 PRYSPRY domain using fluorescence anisotropy and estimated an affinity of ˜19 nM (FIG. 13b). 8 h after their application to cells, tau and BR134 positive puncta were observed inside neurons in complex with TRIM21 (FIG. 13a). Tau that was not labelled with antibody was not found colocalised with bright TRIM21 puncta (FIG. 13a,e,f) consistent with the interaction between antibody Fc region and TRIM21 PRYSPRY domain driving TRIM21 recruitment. The presence of BR134 did not significantly alter the number of tau assemblies found inside neurons (FIG. 13d), suggesting that BR134 does not confer an entry-blocking effect.

### Example 2

### TRIM21 is Essential to Prevent Seeded Tau Aggregation

We next sought to determine if blocking cellular entry of tau assemblies was required for neutralising the seeding activity of tau assemblies. We used a model of seeded tau aggregation in organotypic hippocampal slice cultures (OHSCs) prepared from P301S tau transgenic mice (P301S Tau-Tg; FIG. 1d). OHSCs maintain tau in a soluble state until the addition of tau assemblies, whereupon tau accumulates into insoluble intraneuronal aggregates that are reactive to the phospho-tau specific antibody AT8 (Miller et al., 2021). We observed that pre-treatment of assemblies with AP422, which did not block entry, and mIgG-T, which reduced entry by ˜50%, both reduced seeded aggregation by >90% (FIG. 1e,f). Thus, Abs that do not block entry of tau assemblies can exert neutralisation of seeded aggregation. Moreover, levels of neutralisation by Abs that do block entry are in excess of what would be expected by entry blocking alone.

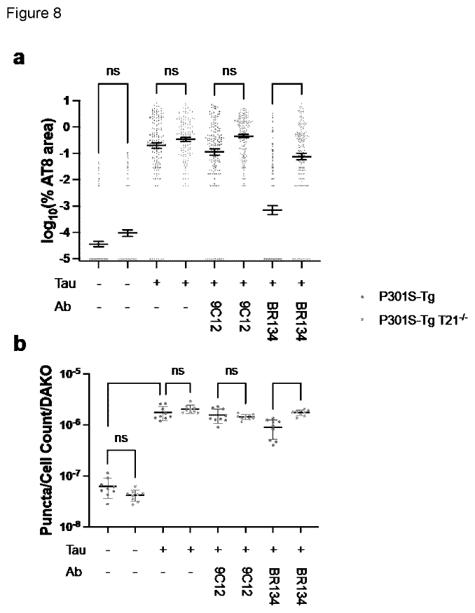

The above results suggest that post-entry mechanisms may be involved in protecting against seeded aggregation in neuronal systems. T21 is a broadly expressed cytosolic Ab receptor that engages antibody-bound proteins and elicits their destruction following activation of its E3 ligase activity (Zeng et al., 2021). To assess the contribution of T21 to tau immunotherapy, we bred a T21-deficient mouse (Yoshimi et al., 2009) on the transgenic P301S human tau background (Allen et al., 2002b) (P301S Tau-Tg T21^{-/-}). We confirmed that OHSCs derived from P301S Tau-Tg T21^{-/-} animals retained normal representation of the major cell types of the CNS and did not express detectable T21 by western blot (FIG. 7). OHSCs derived from both genotypes maintained tau in a native state over 8 weeks in culture and displayed a similar level of seeded aggregation in the absence of Abs (FIG. 1g, 7). However, there was a substantial difference in the observed levels of Ab neutralisation between the genotypes (FIG. 1h, i). Deletion of T21 markedly reduced the ability of AP422 to prevent seeded aggregation. We tested a second antibody, BR134, which is not phosphorylation-dependent and binds to the extreme tau C-terminus (Goedert et al., 1989), and also found its neutralising activity to be highly dependent on T21 (FIG. 8). Furthermore, exogenously supplied assemblies of tau were able to import BR134 to neurons where they were able to contact T21 (FIG. 13). These results demonstrate that neutralisation of seeded aggregation of tau by C-terminal antibodies relies predominantly on intracellular antibody receptor T21.

We next asked whether the activity of antibodies and T21 could inhibit the formation of seed-competent tau species as well as hyperphosphorylated aggregates. We treated OHSCs with tau assemblies in the presence and absence of antibodies as above. OHSC lysates were examined 3 weeks later for the levels of seed-competent species in a sensitive reporter cell line (HEK293 P301S tau-venus (McEwan et al., 2017a)). Untreated OHSCs contained only low levels of tau seeds, whereas those treated with tau assemblies induced substantial levels of seeded aggregation (FIG. 1j). We observed a significant reduction in tau seeds in response to treatment with AP422, but only when T21 was present. In P301S Tau-Tg T21/OHSCs, Abs were unable to reduce the number of seeds that were produced. A similar trend was observed for BR134 (FIG. 8). Taken together these results demonstrate that antibodies and T21 can inhibit the formation of new seed-competent tau assemblies as well as reducing levels of hyperphosphorylated tau inclusions.

### Example 3

### FcγR Activity is not Required for TRIM21-Mediated Protection Against Seeded Aggregation

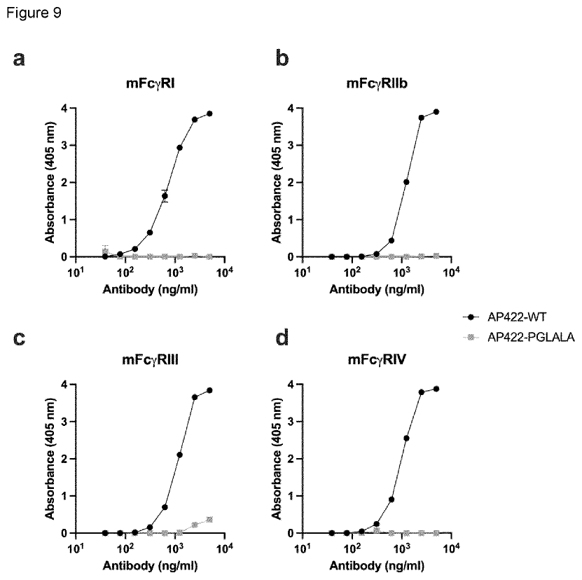

Antibodies can mediate extracellular protection against tau by promoting uptake to microglia via interactions with FcγRs (Andersson et al., 2019; Luo et al., 2015). We therefore examined the contribution of FcγR interaction in preventing seeded aggregation in organotypic slice cultures. We cloned and expressed recombinant AP422 as mouse IgG2a (rAP422) and introduced the Fc amino acid substitutions P329G, L234A and L235A (PGLALA), which abrogate FcγR interactions but maintain interactions with the neonatal Fc receptor FcRn and T21 (Bottermann et al., 2018; Lo et al., 2017). ELISA confirmed that the PGLALA substitutions ablated interactions with mouse FcγRI, FcγRIIB, FcγRIII and FcγRIV (FIG. 9). As a control, we used recombinant mouse IgG2a against ragweed pollen. We found that there was a modest decrease in the protection conferred by rAP422-PGLALA compared to rAP422 (FIG. 1k). However, this effect was considerably smaller than the effects of T21 knockout. This suggests that interactions with cell surface receptors contribute to protection against seeded aggregation but play a comparatively minor role in this system. Together with the genetic knockout data, these results suggest that T21 is primarily responsible for the neutralisation of tau seeding in OHSCs by AP422.

### Example 4

### TRIM21 is Present in Human Neurons and can Exert Viral Neutralisation

Given the above data, an important consideration for tau immunotherapy is the levels of T21 in human neurons, the major site of tau expression and aggregation in Alzheimer's disease and many other tauopathies. We used human iPSC-derived neurons to examine whether T21 is available for Ab-mediated degradation in this setting. Western blot confirmed expression of T21 in human neurons, which was upregulated by treatment with IFNα, an antiviral cytokine known to regulate T21 expression (FIG. 2a) (Mallery et al., 2010). We used neutralisation of an adenovirus type 5 vector expressing GFP under the control of a neuron-specific synapsin promoter (AdV) to determine T21 activity. Treatment of AdV with the anti-hexon mouse monoclonal Ab 9C12 neutralises infection in a T21-dependent manner that can be reversed by heavy chain mutation H433A at the T21 binding interface (McEwan et al., 2012). Using a chimeric mouse-human 9C12 with human Fc domain (rh9C12) (Foss et al., 2016), we observed potent neutralization of infection of AdV infection in human neurons (FIG. 2b). However, rh9C12 with point mutation H433A was almost completely unable to neutralize infection. These results demonstrate that T21 is expressed and active in human neurons, and that the T21 pathway is available for engagement by immunotherapy in this cell type. Furthermore, as in other cell types, neutralisation of AdV in neurons is almost entirely dependent on T21.

### Example 5

Antibodies with Improved TRIM21 Affinity can Increase Neutralisation of Adenovirus Independent of FcγR and FcRn Interactions

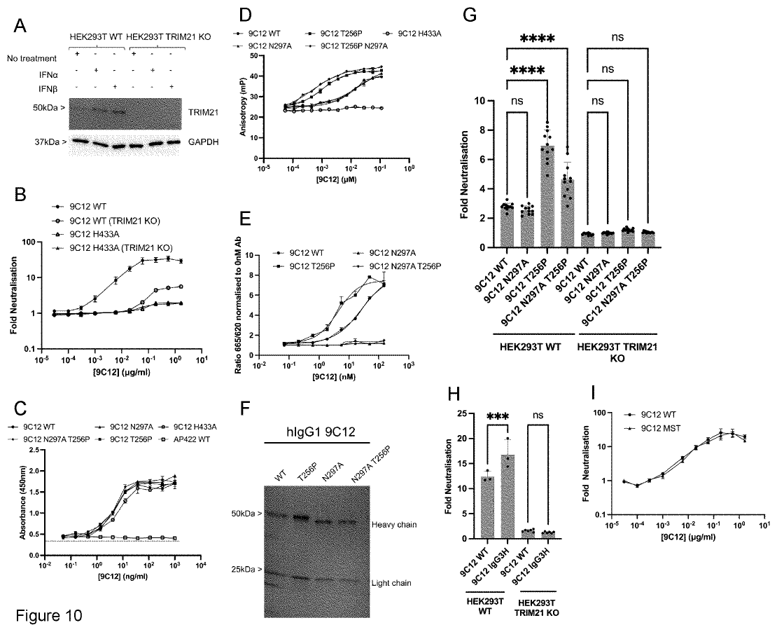

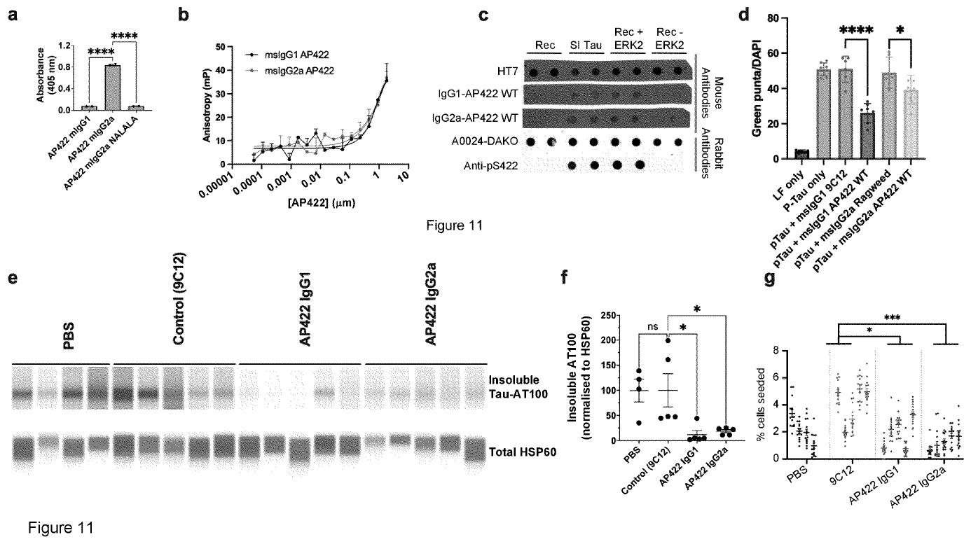

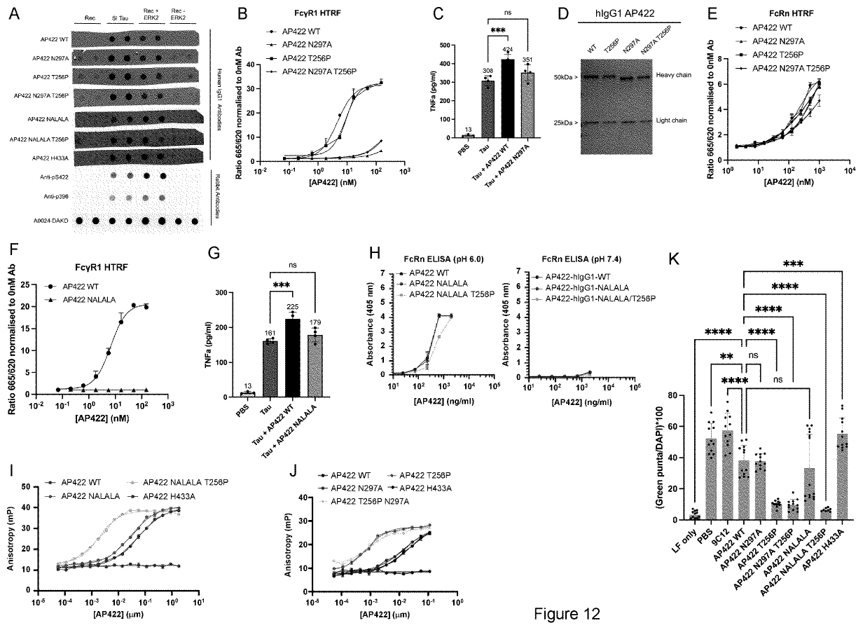

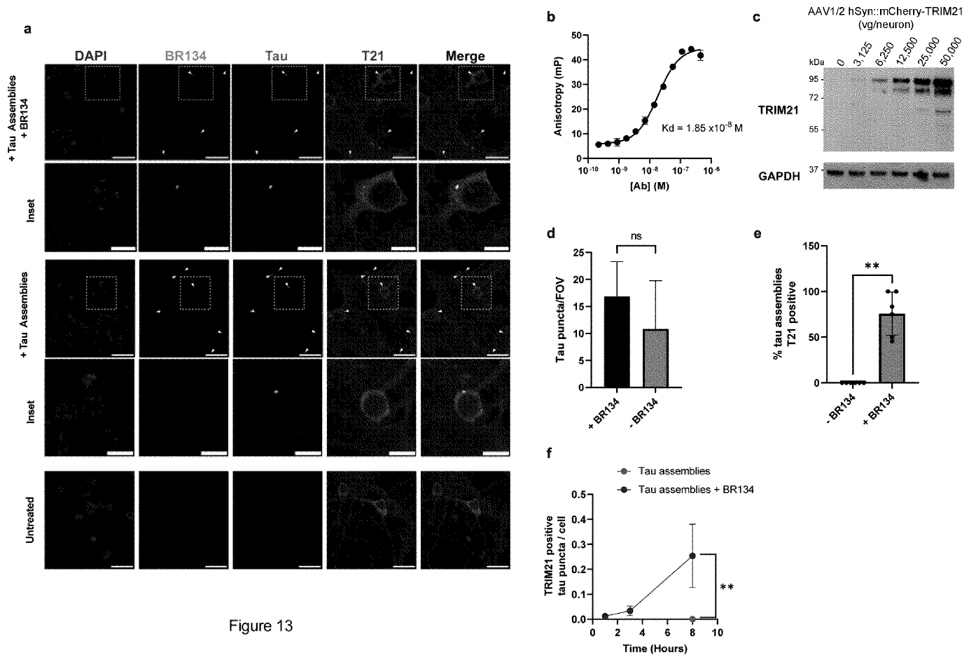

Neutralisation of adenovirus infection by the monoclonal antibody 9C12 is a robust assay for the quantification of TRIM21 activity (McEwan et al., 2012). Adenovirus vector particles encoding GFP are incubated with antibodies at defined concentration before application to cells. Levels of infection are monitored by flow cytometry for percent cells expressing GFP after 24 h. We used cells that were wildtype or were treated with a CRISPR/Cas9 construct targeting the TRIM21 locus (McEwan et al., 2017). Treated cells did not contain detectable TRIM21 by western blot, including in the presence of type I interferon, which increases TRIM21 expression (FIG. 10a). We observed that 9C12, expressed as a mouse-human chimeric antibody with human IgG1 Fc, exerted potent neutralisation in these cells only where TRIM21 was present (FIG. 10b). This demonstrates that intracellular neutralisation is operating via the TRIM21 pathway in this system. As a further confirmation of this, we used missense mutation H433A in the Fc region of 9C12 that prevents interaction with TRIM21 PRYSPRY. This mutation reduced neutralisation, similar to the effect of TRIM21 knockout (FIG. 10b). Thus, interaction with TRIM21 is essential for adenovirus neutralisation by 9C12 in this system.

To examine the contribution of modified TRIM21 and Fc receptor interactions in neutralisation, we next introduced mutations H433A (TRIM21 null), T256P (TRIM21 enhancing), N297A (preventing N-linked glycosylation at this site, thereby reducing FcγR interactions) or double mutant T256P/N297A. We confirmed that the mutated antibodies retained interaction with adenovirus hexon by ELISA (FIG. 10c). We next tested binding of the antibodies to human TRIM21 PRYSPRY using fluorescence anisotropy. Whereas mutation N297A had little effect on PRYSPRY affinity, H433A reduced binding to undetectable levels (FIG. 10d). Conversely, T256P increased affinity by approximately 10-fold (Table 1; FIG. 1d). The double mutant, T256P/N297A had a similarly improved affinity as T256P. Introduction of N297A reduced binding to FcγR compared to wildtype 9C12 or 9C12 T256P in a competitive binding HTRF assay (FIG. 10e) and the lower molecular weight of the N297A mutant heavy chain via denaturing SDS-PAGE (FIG. 10f) is consistent with aglycosylation at this site. Together these results demonstrate that affinity to TRIM21 PRYSPRY can be varied independently of FcγR affinity.

We next investigated the ability of the 9C12 variants to exert neutralisation. We observed that antibodies with enhanced TRIM21 binding (T256P and double mutant T256P/N297A) exerted significantly increased levels of protection against infection (FIG. 10g) after 24 hours. There was no change in neutralisation by the variants in cells that lacked TRIM21. This demonstrates that increasing affinity to TRIM21 PRYSPRY increases neutralisation. We next tested 9C12 expressed with human IgG1 with IgG3 hinge, previously shown to increase TRIM21 activity without modifying affinity (Foss et al., 2022). We likewise found a similar increase in neutralisation that was TRIM21-dependent (FIG. 10h).

To investigate whether antibodies with altered FcRn interactions are altered in their ability to exert neutralisation via TRIM21, we made the MST-YTE mutation, which increases binding to FcRn at acidic pH and improves antibody half-life (Vaccaro et al., 2005). We observed that this mutation did not affect the ability of 9C12 to neutralise infection (FIG. 10i). This suggests that FcRn interactions may be improved without impacting TRIM21 activity.

TABLE 1
Dissociation constant (Kd) for 9C12
Antibodies to Human TRIM21 PRYSPRY

| Antibody | Kd (nM) |
|---|---|
| 9C12 WT | 11.39 |
| 9C12 N297A | 24.50 |
| 9C12 T256P | 1.00 |
| 9C12 N297A T256P | 0.4669 |
| 9C12 H433A | No binding |

### Example 6

### Trim21 is Required for Protection In Vivo

We next investigated the role of T21 in an in vivo model of tau pathology. In P301S-Tg mice, incipient tau pathology can be detected by immunoreactivity to phospho-tau in lumbar spinal sections from 1 month followed by amplification of signal until 7 months (Macdonald et al., 2019). We found that sarkosyl insoluble tau and seed-competent species increased dramatically between 20 and 80 days of age (FIG. 2c-e). No seed-competent species were detected in non-transgenic C57BL/6 mouse spines (FIG. 2e), indicating that seeding activity arises from transgenic tau. We therefore asked whether Abs could reduce incipient tau pathology by passive transfer into P301S Tau-Tg T21^{+/+} and P301S Tau-Tg T21^{-/-} mice. Mice were treated with AP422, control antibody 9C12 or buffer only (PBS) by weekly intraperitoneal (i.p.) injection. Western blot revealed a ˜75% reduction in insoluble tau levels following AP422 treatment in T21^{+/+} animals (FIG. 2f,g). However, no Ab protection was observed in T21/animals or when the control Ab 9C12 was used. We further examined levels of seed-competent tau species in these preparations and observed a significant T21-dependent reduction following AP422 treatment (FIG. 2h). Of note, the protection against sarkosyl insoluble tau accumulation was of greater magnitude than the reduction in the generation of new seed competent species. This is of interest as we have recently shown that T21 is activated by a stoichiometric threshold of antibodies (Zeng et al., 2021). Given that seeds can be of low stoichiometric value, potentially even monomers (Hou et al., 2021; Mirbaha et al., 2018), our findings suggest that fibrillar tau may represent a better substrate for T21 degradation than small tau assemblies. In summary, the results show that antibody can reduce levels of incipient tau aggregation in the mouse brain in a manner that requires T21.

We next sought to establish the involvement of T21 in a longer-term Ab treatment regimen. We compared treatment with AP422, which displays no entry blocking, and mIgG-T, which binds at multiple sites throughout tau and reduces entry of tau to the cytosol of primary neurons. P301S Tau-Tg T21^{+/+} and P301S Tau-Tg T21^{-/-} mice were treated for 17 weeks by weekly administration of Abs to the periphery and levels of total and sarkosyl insoluble tau in the brain were examined by western blot. Both antibodies conferred protection against accumulation of sarkosyl insoluble tau and AT100-positive tau in T21^{+/+} animals (FIG. 3a-d). Protection was strongest for mIgG-T, which provided a ˜90% reduction in sarkosyl insoluble tau, compared to AP422 which provided ˜60% protection. However, in T21^{-/-} mice, neither Ab protected against tau pathology. The fact that mIgG-T did not protect in T21/mice suggests that blocking of entry is not the main correlate of protection in vivo, where protection is reliant on T21. These results demonstrate that T21 expression is required for protection in a model of chronic immunotherapy for both an entry-blocking Ab and a non-entry blocking Ab.

### Example 7

Use of Antibody Classes with Reduced FcγR Affinity can Preserve Protection

We investigated how selection of IgG subclasses with different affinities to FcγR were able to protect against tau pathology during passive immunotherapy. We expressed AP422 as mouse IgG1, which has low affinity for FcγRs, or as mouse IgG2a, which has higher affinity. We confirmed that AP422-IgG2a had the ability to interact with CD64 (FcγR1), whereas AP422-IgG1 or AP422-IgG2a-NALALA did not (FIG. 11a). Both AP422-IgG1 and AP422-IgG2a were able to bind to mouse TRIM21 PRYSPRY domain to a similar extent (FIG. 11b) and both were able to interact with pTau by dot blot. In tau seeding experiments in HEK293 cells both antibody subclasses were able to confer protection relative to control antibodies, 9C12 mIgG1 or anti-ragweed mIgG2a (FIG. 11d). Passive transfer of AP422 IgG1 vs IgG2a demonstrated that both antibodies retained the ability to confer protection against AT100-reactive insoluble tau and against seed-competent tau species (FIG. 11e-g). IgG subclass with low FcγR interactions can therefore be used for effective immunotherapy provided they retain interactions with TRIM21 PRYSPRY. This suggests that human IgG4 which, like mIgG1, has lower affinity FcγR interactions, may be suitable for immunotherapy via TRIM21 while minimising pro-inflammatory effects.

### Example 8

### Experimental Introduction of Antibodies to Cells Permits Intracellular Neutralisation of Seeded Aggregation.

Antibodies may be introduced into cells in order to degrade proteins via the technique Trim-Away (Clift et al., 2017). Introduction can be using microinjection, or electroporation in order to deliver sufficient antibody to the cell to elicit TRIM21-mediated degradation of proteins that are bound by the antibody in question. We asked whether seeded aggregation of tau could be inhibited in this manner. AP422 or a control antibody were electroporated into HEK293 cells expression P301S tau-venus prior to addition of tau seeds, which were delivered into cells by lipofectamine. AP422 exerted a potent block to seeded aggregation, demonstrating that antibodies can exert neutralisation of seeding post-entry (FIG. 4). This extends the previous finding that tau-antibody complexes can be co-delivered into cells and protect in the intracellular domain (McEwan et al., 2017a).

### Example 9

Deglycosylation of Antibodies does not Prevent TRIM21 Activity

The prevention or removal of N-linked glycosylation of antibodies at N297 reduces or prevents interaction with FcγRs. We asked whether TRIM21 activity was similarly affected by the deglycosylation of antibody. By examining levels of TRIM21 activity, namely NF-κB activation and adenovirus neutralisation, we observed that deglycosylation did not prevent TRIM21-mediated activity (FIG. 5). We deglycosylated human IgG, which possesses a conserved N-linked glycosylation site at asparagine 297, using the following enzymes: neuraminidase (Neura), peptide N-glycosidase F (PNGase F) or a deglycosylation mix available from New England Biolabs (DG mix). After treatment, antibodies were purified on a protein G column. Antibodies were then incubated with AdV, and used for neutralisation and signalling assays. Treatment with these enzymes had no effect on TRIM21-dependent functions of intracellular neutralisation and signalling (FIG. 5). Deglycosylation was verified by running samples under reducing conditions by SDS-PAGE and mass spectrometry to confirm absence of glycans on peptide EEQYNSTFR (293-301 of human IgG1). We found that both PNGase F and DG mix were both able to remove the full glycan chain from this peptide. Thus, in the present invention, removal of antibody glycans by mutation of N297, or during or after antibody synthesis can be employed to reduce FcγRs without unduly impacting on TRIM21 effector functions.

### Example 10. Antibodies with Improved TRIM21 Affinity can Increase Neutralisation of Tau Independent of FcγR and FcRn Interactions

We next examined whether antibodies with increased TRIM21 binding characteristics could exert more potent neutralisation against seeded tau aggregation. We expressed the anti-phospho tau antibody AP422 as a mouse-human chimera with human Fc region. We introduced mutations T256P, N297A and double mutant T256P/N297A as previous. We first confirmed that all mutants retained binding activity against phosphorylated tau (FIG. 12a). Introduction of N297A reduced binding to FcγR1 compared to wildtype or AP422 T256P in a competitive binding HTRF assay (FIG. 12b). As expected, introduction of N297A reduced the inflammatory effects of tau:antibody immune complexes in human microglia (FIG. 12c). The heavy chain of antibody bearing N297A mutant was observed to run at a lower molecular weight compared to unmodified antibody via denaturing SDS-PAGE (FIG. 12d) consistent with aglycosylation at this site. Furthermore, introducing these mutations did not ablate human FcRn binding, as measured in a competitive binding HTRF assay (FIG. 12e).

We noted that a residual amount of interaction with FcγR was detectable in N297A, and therefore additionally produced AP422 with the NALALA mutations, which ablated all detectable interaction with FcγR, and similarly reduced the pro-inflammatory effects of antibody (FIG. 12e,f). However, like N297A, NALALA did not prevent interaction with TRIM21 PRYPSRY (FIG. 12g). Introducing these mutations also did not prevent human FcRn binding at pH 6, as measured using human FcRn by ELISA (FIG. 12h).

The introduction of N297A or NALALA did not substantially change affinity of antibodies to TRIM21 PRYPSRY (FIG. 12i,j). However T256P, either alone or in combination with N297A or NALALA substantially increased affinity to PRYSPRY (Table 2,3; FIG. 12i,j). Thus, the affinity of anti-tau antibodies to TRIM21 PRYSPRY can be varied independently of FcγR affinity.

We next tested the modified antibodies in the neutralisation of tau seeding. We used HEK293 cells expressing P301S tau-venus which respond to the presence of tau assemblies by the formation of intracellular puncta that can be quantified by high-content microscopy We previously demonstrated that antibody neutralisation of seeded aggregation in the presence of lipofectamine is dependent on TRIM21 in this cell type (McEwan et al., 2017). We observed that AP422 _{WT} was able to exert a modest level of protection against neutralisation (FIG. 12k). However, increasing affinity to TRIM21 by introduction of T256P substantially increased neutralisation. Addition of N297A, either alone, or in combination with T256P, had no effect on neutralisation. Mutation H433A, which reduced the affinity of AP422 to TRIM21 PRYSPRY (FIG. 12i, j), prevented neutralisation, consistent with a central role for TRIM21-mediated neutralisation in this system. These results were further confirmed using triple mutation NALALA, which has no detectable interaction with FcγRs, and likewise had no impact on neutralisation (FIG. 12i,k). These results demonstrate that affinity to TRIM21 is important in overall levels of antibody-mediated protection against seeded tau aggregation. Reducing antibody affinity to TRIM21, for example using H433A, prevents neutralisation, while increasing affinity, for example using T256P, can enhance it. Further, the results demonstrate that reducing affinity for FcγRs can reduce inflammatory responses to immune complexes by human microglia, whilst preserving TRIM21-mediated effector function.

TABLE 2
Dissociation constant (Kd) for recombinant
mouse-human chimeric AP422 antibodies with
human Fc region with human TRIM21 PRYSPRY

| Antibody | Kd (nM) |
|---|---|
| AP422 WT | 23 |
| AP422 N297A | 14 |
| AP422 T256P | 0.7 |
| AP422 N297A T256P | 0.9 |
| AP422 H433A | No binding |

TABLE 3
Dissociation constant (Kd) for recombinant
mouse-human chimeric AP422 antibodies with
human Fc region with human TRIM21 PRYSPRY

| Antibody | Kd (nM) |
|---|---|
| AP422 WT | 53 |
| AP422 NALALA | 29 |
| AP422 NALALA T256P | 0.1 |
| AP422 H433A | No binding |

### REFERENCES

Adolfsson, O., Pihlgren, M., Toni, N., Varisco, Y., Buccarello, A. L., Antoniello, K., Lohmann, S., Piorkowska, K., Gafner, V., Atwal, J. K., et al. (2012). An Effector-Reduced Anti-B-Amyloid (AB) Antibody with Unique AB Binding Properties Promotes Neuroprotection and Glial Engulfment of AB. J. Neurosci. 32, 9677-9689.
Allen, B., Ingram, E., Takao, M., Smith, M. J., Jakes, R., Virdee, K., Yoshida, H., Holzer, M., Craxton, M., Emson, P. C., et al. (2002a). Abundant Tau Filaments and Nonapoptotic Neurodegeneration in Transgenic Mice Expressing Human P301S Tau Protein. J Neurosci 22, 9340-9351.
Allen, B., Ingram, E., Takao, M., Smith, M. J., Jakes, R., Virdee, K., Yoshida, H., Holzer, M., Craxton, M., Emson, P. C., et al. (2002b). Abundant tau filaments and nonapoptotic neurodegeneration in transgenic mice expressing human P301S tau protein. J. Neurosci. 22, 9340-9351.
Andersson, C. R., Falsig, J., Stavenhagen, J. B., Christensen, S., Kartberg, F., Rosenqvist, N., Finsen, B., and Pedersen, J. T. (2019). Antibody-mediated clearance of tau in primary mouse microglial cultures requires Fcγ-receptor binding and functional lysosomes. Sci Rep 9.
Asuni A A, Boutajangout A, Quartermain D, Sigurdsson E M. Immunotherapy targeting pathological tau conformers in a tangle mouse model reduces brain pathology with associated functional improvements. J Neurosci. (2007) 27:9115-29. doi: 10.1523/JNEUROSCI.2361-07.2007
Bottermann, M., Foss, S., van Tienen, L. M., Vaysburd, M., Cruickshank, J., O'Connell, K., Clark, J., Mayes, K., Higginson, K., Hirst, J. C., et al. (2018). TRIM21 mediates antibody inhibition of adenovirus-based gene delivery and vaccination. Proc. Natl. Acad. Sci. U.S.A.
Bottermann M, Foss S, Caddy SL, Clift D, van Tienen L M, Vaysburd M, et al. Complement C4 prevents viral infection through capsid inactivation. Cell Host Microbe. (2019) 25:617-29.e7. doi: 10.1016/j.chom.2019.02.016
Bottermann M, James L C. Intracellular antiviral immunity. Adv Virus Res. (2018) 100:309-54. doi: 10.1016/bs.aivir.2018.01.002
Bournazos S, Klein F, Pietzsch J, Seaman M S, Nussenzweig M C, Ravetch J V. Broadly neutralizing anti-HIV-1 antibodies require Fc effector functions for in vivo activity. Cell. (2014) 158:1243-53. doi: 10.1016/j.cell.2014.08.023
Chai X, Wu S, Murray T K, Kinley R, Cella C V, Sims H, et al. Passive immunization with anti-Tau antibodies in two transgenic models: reduction of Tau pathology and delay of disease progression. J Biol Chem. (2011) 286:34457-67. doi: 10.1074/jbc.M111.229633
Clift, D., McEwan, W. A., Labzin, L. I., Konieczny, V., Mogessie, B., James, L. C., and Schuh, M. (2017). A Method for the Acute and Rapid Degradation of Endogenous Proteins. Cell 171, 1692-1706.e18.
DiLillo D J, Tan G S, Palese P, Ravetch J V. Broadly neutralizing hemagglutinin stalk-specific antibodies require FcgammaR interactions for protection against influenza virus in vivo. Nat Med. (2014) 20:143-51. doi: 10.1038/nm.3443
Fernandopulle, M. S., Prestil, R., Grunseich, C., Wang, C., Gan, L., and Ward, M. E. (2018). Transcription Factor-Mediated Differentiation of Human iPSCs into Neurons. Curr Protoc Cell Biol 79, e51.
Foss, S., Watkinson, R. E., Grevys, A., McAdam, M. B., Bern, M., Høydahl, L. S., Dalhus, B., Michaelsen, T. E., Sandlie, I., James, L. C., et al. (2016). TRIM21 Immune Signaling Is More Sensitive to Antibody Affinity Than Its Neutralization Activity. J. Immunol. 196, 3452-3459.
S. Foss, A. Jonsson, M. Bottermann, R. Watkinson, H. E. Lode, M. B. McAdam, T. E. Michaelsen, I. Sandlie, L. C. James, J. T. Andersen, Potent TRIM21 and complement-dependent intracellular antiviral immunity requires the IgG3 hinge. Sci Immunol. 7, eabj1640 (2022).
Frost B, Jacks R L, Diamond M I. Propagation of tau misfolding from the outside to the inside of a cell. J Biol Chem. (2009) 284:12845-52. doi: 10.1074/jbc.M808759200
Goedert, M., and Jakes, R. (1990). Expression of separate isoforms of human tau protein: correlation with the tau pattern in brain and effects on tubulin polymerization. EMBO J 9, 4225-4230.
Goedert, M., Spillantini, M. G., Jakes, R., Rutherford, D., and Crowther, R. A. (1989). Multiple isoforms of human microtubule-associated protein tau: sequences and localization in neurofibrillary tangles of Alzheimer's disease. Neuron 3, 519-526.
Goedert, M., Spillantini, M. G., Cairns, N. J., and Crowther, R. A. (1992). Tau proteins of alzheimer paired helical filaments: Abnormal phosphorylation of all six brain isoforms. Neuron 8, 159-168.
Guo J L, Lee V M. Seeding of normal Tau by pathological Tau conformers drives pathogenesis of Alzheimer-like tangles. J Biol Chem. (2011) 286:15317-31. doi: 10.1074/jbc.M110.209296
Hasegawa, M., Jakes, R., Crowther, R. A., Lee, V. M., Ihara, Y., and Goedert, M. (1996). Characterization of mAb AP422, a novel phosphorylation-dependent monoclonal antibody against tau protein. FEBS Lett 384, 25-30.
Hillen, M. R., Urso, K., Koppe, E., Lopes, A. P., Blokland, S. L. M., Pandit, A., Slocombe, T., van Maurik, A., van Roon, J. A. G., and Radstake, T. R. D. J. (2020). Autoantigen TRIM21/Ro52 is expressed on the surface of antigen-presenting cells and its enhanced expression in Sjögren's syndrome is associated with B cell hyperactivity and type I interferon activity. RMD Open 6, e001184.
Holmes, B. B., DeVos, S. L., Kfoury, N., Li, M., Jacks, R., Yanamandra, K., Ouidja, M. O., Brodsky, F. M., Marasa, J., Bagchi, D. P., et al. (2013). Heparan sulfate proteoglycans mediate internalization and propagation of specific proteopathic seeds. Proc. Natl. Acad. Sci. U.S.A. 110, E3138-3147.
Hou, Z., Chen, D., Ryder, B. D., and Joachimiak, L. A. (2021). Biophysical properties of a tau seed. Sci Rep 11, 13602.
Klasse P J, Sattentau Q J. Occupancy and mechanism in antibody-mediated neutralization of animal viruses. J Gen Virol. (2002) 83 (Pt 9): 2091-108. doi: 10.1099/0022-1317-83-9-2091
Kondo, A., Shahpasand, K., Mannix, R., Qiu, J., Moncaster, J., Chen, C.-H., Yao, Y., Lin, Y.-M., Driver, J.A., Sun, Y., et al. (2015). Antibody against early driver of neurodegeneration cis P-tau blocks brain injury and tauopathy. Nature 523, 431-436.
Leung D T, Tam F C, Ma C H, Chan P K, Cheung J L, Niu H, et al. Antibody response of patients with severe acute respiratory syndrome (SARS) targets the viral nucleocapsid. J Infect Dis. (2004) 190:379-86. doi: 10.1086/422040
Leuzy, A., Chiotis, K., Lemoine, L., Gillberg, P.-G., Almkvist, O., Rodriguez-Vieitez, E., and Nordberg, A. (2019). Tau PET imaging in neurodegenerative tauopathies-still a challenge. Mol Psychiatry 24, 1112-1134.
Lo, M., Kim, H. S., Tong, R. K., Bainbridge, T. W., Vernes, J.-M., Zhang, Y., Lin, Y. L., Chung, S., Dennis, M. S., Zuchero, Y. J. Y., et al. (2017). Effector-attenuating Substitutions That Maintain Antibody Stability and Reduce Toxicity in Mice. J. Biol. Chem. 292, 3900-3908.
Luo, W., Liu, W., Hu, X., Hanna, M., Caravaca, A., and Paul, S. M. (2015). Microglial internalization and degradation of pathological tau is enhanced by an anti-tau monoclonal antibody. Sci Rep 5.
Macdonald, J. A., Bronner, I. F., Drynan, L., Fan, J., Curry, A., Fraser, G., Lavenir, I., and Goedert, M. (2019). Assembly of transgenic human P301S Tau is necessary for neurodegeneration in murine spinal cord. Acta Neuropathol Commun 7, 44.
Mallery, D. L., McEwan, W. A., Bidgood, S. R., Towers, G. J., Johnson, C. M., and James, L. C. (2010). Antibodies mediate intracellular immunity through tripartite motif-containing 21 (TRIM21). Proc. Natl. Acad. Sci. U.S.A. 107, 19985-19990.
McEwan, W. A., Hauler, F., Williams, C. R., Bidgood, S. R., Mallery, D. L., Crowther, R. A., and James, L. C. (2012). Regulation of virus neutralization and the persistent fraction by TRIM21. J. Virol. 86, 8482-8491.
McEwan W A, Tam J C, Watkinson R E, Bidgood S R, Mallery D L, James L C. Intracellular antibody-bound pathogens stimulate immune signaling via the Fc receptor TRIM21. Nat Immunol. (2013) 14:327-36. doi: 10.1038/ni.2548
McEwan, W. A., Falcon, B., Vaysburd, M., Clift, D., Oblak, A. L., Ghetti, B., Goedert, M., and James, L. C. (2017). Cytosolic Fc receptor TRIM21 inhibits seeded tau aggregation. Proc. Natl. Acad. Sci. U.S.A. 114, 574-579.
Miller, L. V. C., Mukadam, A. S., Durrant, C. S., Vaysburd, M. J., Katsinelos, T., Tuck, B. J., Sanford, S., Sheppard, O., Knox, C., Cheng, S., et al. (2021). Tau assemblies do not behave like independently acting prion-like particles in mouse neural tissue. Acta Neuropathol Commun 9, 1-16.
Miranda, M. E., Tseng, C. E., Rashbaum, W., Ochs, R. L., Casiano, C. A., Di Donato, F., Chan, E. K., and Buyon, J. P. (1998). Accessibility of SSA/Ro and SSB/La antigens to maternal autoantibodies in apoptotic human fetal cardiac myocytes. J Immunol 161, 5061-5069.
Mirbaha, H., Chen, D., Morazova, O. A., Ruff, K. M., Sharma, A. M., Liu, X., Goodarzi, M., Pappu, R. V., Colby, D. W., Mirzaei, H., et al. (2018). Inert and seed-competent tau monomers suggest structural origins of aggregation. ELife 7, e36584.
Ng, P. M. L., Kaliaperumal, N., Lee, C. Y., Chin, W. J., Tan, H. C., Au, V. B., Goh, A. X.-H., Tan, Q. W., Yeo, D. S. G., Connolly, J. E., et al. (2019). Enhancing Antigen Cross-Presentation in Human Monocyte-Derived Dendritic Cells by Recruiting the Intracellular Fc Receptor TRIM21. J Immunol 202, 2307-2319.
Norderhaug, L., Olafsen, T., Michaelsen, T. E., and Sandlie, I. (1997). Versatile vectors for transient and stable expression of recombinant antibody molecules in mammalian cells. J Immunol Methods 204, 77-87.
Odendall C, Kagan JC. Activation and pathogenic manipulation of the sensors of the innate immune system. Microbes Infect. (2017) 19:229-37. doi: 10.1016/j.micinf.2017.01.003
Rakebrandt N, Lentes S, Neumann H, James LC, Neumann-Staubitz P. Antibody- and TRIM21-dependent intracellular restriction of Salmonella enterica. Pathog Dis. (2014) 72:131-7. doi: 10.1111/2049-632X. 12192
Randow F, MacMicking J D, James L C. Cellular self-defense: how cellautonomous immunity protects against pathogens. Science. (2013) 340:701-6. doi: 10.1126/science. 1233028
Rauch, J. N., Luna, G., Guzman, E., Audouard, M., Challis, C., Sibih, Y. E., Leshuk, C., Hernandez, I., Wegmann, S., Hyman, B. T., et al. (2020). LRP1 is a master regulator of tau uptake and spread. Nature 580, 381-385.
Rubinsztein, D.C. (2006). The roles of intracellular protein-degradation pathways in neurodegeneration.
Sankaranarayanan S, Barten D M, Vana L, Devidze N, Yang L, Cadelina G, et al. Passive immunization with phospho-tau antibodies reduces tau pathology and functional deficits in two distinct mouse tauopathy models. PLOS ONE. (2015) 10: e0125614. doi: 10.1371/journal.pone.0125614
Schrader J W, McLean G R. Location, location, timing: analysis of cytomegalovirus epitopes for neutralizing antibodies. Immunol Lett. (2007) 112:58-60. doi: 10.1016/j.imlet.2007.07.001
Shi, Y., Zhang, W., Yang, Y., Murzin, A. G., Falcon, B., Kotecha, A., van Beers, M., Tarutani, A., Kametani, F., Garringer, H. J., et al. (2021). Structure-based classification of tauopathies. Nature 598, 359-363.
Silva, M. C., Ferguson, F. M., Cai, Q., Donovan, K. A., Nandi, G., Patnaik, D., Zhang, T., Huang, H.-T., Lucente, D. E., Dickerson, B. C., et al. (2019). Targeted degradation of aberrant tau in frontotemporal dementia patient-derived neuronal cell models. ELife 8, e45457.
Sumida S M, Truitt D M, Lemckert A A, Vogels R, Custers J H, Addo M M, et al. Neutralizing antibodies to adenovirus serotype 5 vaccine vectors are directed primarily against the adenovirus hexon protein. J Immunol. (2005) 174:7179-85. doi: 10.4049/jimmunol. 174.11.7179
Tam JC, Bidgood SR, McEwan W A, James LC. Intracellular sensing of complement C3 activates cell autonomous immunity. Science. (2014) 345:1256070. doi: 10.1126/science. 1256070
Tuck, B. J., Katsinelos, T., Miller, L. V. C., Cheng, S., Vaysburd, M. J., Knox, C., Tredgett, L., James, L. C., and McEwan, W. A. (2021). Tau assemblies enter the cytosol of neurons in a cholesterol sensitive manner. BioRxiv 2021.06.21.449238.
C. Vaccaro, J. Zhou, R. J. Ober, E. S. Ward, Engineering the Fc region of immunoglobulin G to modulate in vivo antibody levels. Nat Biotechnol. 23, 1283-1288 (2005).
Washer, S. J., Perez-Alcantara, M., Chen, Y. et al. Single-cell transcriptomics defines an improved, validated monoculture protocol for differentiation of human iPSC to microglia. Sci Rep 12, 19454 (2022). https://doi.org/10.1038/s41598-022-23477-2
Yanamandra K, Kfoury N, Jiang H, Mahan T E, Ma S, Maloney S E, et al. Anti-tau antibodies that block tau aggregate seeding in vitro markedly decrease pathology and improve cognition in vivo. Neuron. (2013) 80:402-14. doi: 10.1016/j.neuron.2013.07.046
Yoshida, H., Hastie, C. J., McLauchlan, H., Cohen, P., and Goedert, M. (2004). Phosphorylation of microtubule-associated protein tau by isoforms of c-Jun N-terminal kinase (JNK). Journal of Neurochemistry 90, 352-358.
Yoshimi, R., Chang, T.-H., Wang, H., Atsumi, T., Morse, H. C., and Ozato, K. (2009). Gene disruption study reveals a non-redundant role for TRIM21/Ro52 in NF-κB-dependent cytokine expression in fibroblasts. J Immunol 182, 7527-7538.
Zeng, J., Santos, A. F., Mukadam, A. S., Osswald, M., Jacques, D. A., Dickson, C.F., McLaughlin, S. H., Johnson, C. M., Kiss, L., Luptak, J., et al. (2021). Target-induced clustering activates Trim-Away of pathogens and proteins. Nat Struct Mol Biol 28, 278-289.
Zilkova, M., Nolle, A., Kovacech, B., Kontsekova, E., Weisova, P., Filipcik, P., Skrabana, R., Prcina, M., Hromadka, T., Cehlar, O., et al. (2020). Humanized tau antibodies promote tau uptake by human microglia without any increase of inflammation. Acta Neuropathologica Communications 8, 74.

1. A method of treating neurodegenerative disease in the cytoplasm of a neuronal cell in a subject, comprising administering a ligand comprising a first binding moiety which binds to tau assemblies, and a second binding moiety which is bound by TRIM21, wherein the ligand is administered extracellularly.

2. The method of claim 1, wherein ligand is administered intravenously to a subject.

3. The method of claim 1, which is selected from a polypeptide, a structured polypeptide, a small molecule and an immunoglobulin, optionally wherein the immunoglobulin is an antibody, further optionally wherein the antibody comprises a variable domain antigen binding region which binds to tau assemblies, and an Fc region which is bound by TRIM21.

4-5. (canceled)

6. The method of claim 3, in which the ability to bind to FcγR and/or complement (C1q) has been reduced or eliminated.

7. The method of claim 3, wherein the ability of the Fc domain to bind to TRIM21 has been increased, relative to an unmodified antibody Fc domain.

8. The method of claim 3 to 7, wherein recycling via FcRn is enhanced.

9. The method of claim 3, wherein the antibody is modified to reduce glycosylation; preferably, wherein the antibody is modified by the mutation N297A.

10. The method of claim 3, wherein the ability of the Fc domain to bind to TRIM21 has been increased, relative to an unmodified antibody Fc domain, by introducing the T256P mutation and/or by modification of the antibody hinge region, such as by replacement of the hinge region with an IgG3 hinge region.

11. The method of claim 3, wherein recycling via FcRn is enhanced by insertion of one, two, three or four mutations selected from the group consisting of M252Y, S254T, T256E, H433K and N434F, or M428L/N434S or T256D/T307Q (DQ) or T256D/T307W (DW) or M252Y/T256D (YD) or T307Q/Q311V/A383V or T256D/H286D/T307R/Q311V/A378A or L309D/Q311H/N434S (DHS) or M252Y/S254T/T256E (MST-YTE) into the IgG1 Fc domain.

12. The method of claim 3, wherein FcγR binding is reduced by causing a loss of glycosylation, for example by introducing the mutation N297A, and/or by introducing mutations selected from the group consisting of P329G, L234A and L235A (PGLALA), L234F/L235E/P331S (FES), L234F/L235E/D265A (FEA), L234A/L235A (LALA) and N297A/L234A/L235A (NALALA) into the IgG1 Fc domain, or said Fc domain is derived from an immunoglobulin class or isotype that has reduced affinity for Fc γ receptors or complement and their derivatives that further ablate binding (eg IgG4-PE S228P/L235E).

13. The method of claim 3, which has been modified for increased intracellular stability.

14. A method for degrading a target in a cell, comprising administering to the cell an antibody specific for the target, said antibody being modified to increase binding to Trim21 in comparison to an unmodified antibody, by introducing a mutation selected from T256P and N297A, or a combination of T256P and N297A; and/or by modification of the antibody hinge region, such as by replacement of the hinge region with an IgG3 hinge region.

15. The method according to claim 14, wherein the target is a molecule which can transit into a cell when attached to an antibody, for example a target selected from the group consisting of viruses, protein aggregates, tau, alpha-synuclein, TDP43 and SOD1, optionally wherein the target is a misfolded or aggregated form of a protein.

16. (canceled)

17. The method according to claim 14, comprising administering said antibody extracellularly and allowing it to bind to the target, such that it is introduced into the cell in association with the target.

18. The method according to, claim 14 wherein the antibody is a ligand according to claim 9.

19. A complex comprising an anti-tau antibody in which the ability to bind to FcγR and/or complement (C1q) has been reduced or eliminated, bound to tau protein.

20. The complex according to claim 19, wherein the antibody is an antibody according to claim 12.

21. The complex according to claim 19, wherein the antibody has been modified for increased intracellular stability.

22. A cell comprising within its cytoplasm a complex according to claim 19, optionally wherein the cell is a neuronal cell.

23. (canceled)

24. A method for treating or preventing a viral disease, a protein aggregation disorder or tau pathology in a subject, comprising administering to the subject a ligand comprising a first binding moiety which binds to tau assemblies, and a second binding moiety which is bound by TRIM21.
